(19) United States
(12) Patent Application Publication
(10) Pub. No.: US 2024/0148511 A1
(43) Pub. Date: May 9, 2024

(54) UNIVERSAL PAYLOAD FOR INTEGRATION WITH EACH OF A PLURALITY OF DIFFERENT ORTHOPEDIC IMPLANTS HAVING A PAYLOAD RECEPTACLE

(71) Applicant: Canary Medical Inc., Vancouver (CA)

(72) Inventors: Michael Boothby, San Diego, CA (US); John Ray, Indian Creek, IL (US); Matthew Sturtevant, San Diego, CA (US)

(21) Appl. No.: 18/379,564

(22) Filed: Oct. 12, 2023

Related U.S. Application Data
(63) Continuation-in-part of application No. PCT/US2023/017972, filed on Apr. 7, 2023.
(60) Provisional application No. 63/328,568, filed on Apr. 7, 2022.

(51) Int. Cl. A61F 2/38; A61B 5/00; A61B 5/11; A61F 2/36; A61F 2/40

(52) U.S. Cl. CPC A61F 2/389; A61B 5/1126; A61B 5/6847; A61F 2/3662; A61F 2/4059; A61B 2562/0219; A61F 2002/3067

(57) ABSTRACT

A universal payload may be integrated with each of a plurality of different orthopedic implants configured to replace or functionally supplement a natural joint of a body. In one aspect, a universal payload includes an electronics assembly, a housing enclosing the electronics assembly, and a payload coupling feature configured to couple to each of a plurality of different orthopedic implants. The electronics assembly includes at least one sensor and a controller having at least two joint-specific modules. Each joint-specific module is respectively configured to collect kinematic data resulting from movement of a respective specific joint sensed by the at least one sensor. In another aspect, a universal payload includes an electronics assembly comprising at least one sensor and a housing enclosing the electronics assembly. The universal payload has a payload form factor that is configured to be at least partially inserted into a payload receptacle in each of a plurality of different orthopedic implants.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### TECHNICAL FIELD

The present disclosure relates generally to orthopedic implants, and more particularly, to a universal payload configured for integration with each of a plurality of different types of orthopedic implants having a payload receptacle.

### BACKGROUND

Joint replacement systems, such as knee arthroscopy systems, shoulder arthroscopy systems, and hip arthroscopy systems, include implant structures or components of assorted sizes and configurations. Some of these implant structures or components, referred to herein as orthopedic implants, may include electronics and sensors that are built into the implants during manufacturing. These electronics and sensors may be used to collect data relevant to the orthopedic implant. Given the variety in sizes and configurations of these orthopedic implants, it is desirable to have a component with electronics and sensors that can be integrated with each of a plurality of different orthopedic implants—independent of the manufacturing of these implants. For example, it may be desirable to have the ability to integrate such a component into any select one of variety of orthopedic implant of different sizes and/or configurations at the time of surgical implant.

### SUMMARY

Briefly stated, the present disclosure relates to universal payloads (also referred to as universal cartridges) that may be integrated with each of a plurality of different orthopedic implants configured to replace or functionally supplement a natural joint of a body. In one aspect, a universal payload includes an electronics assembly, a housing enclosing the electronics assembly, and a payload coupling feature configured to couple to each of a plurality of different orthopedic implants. The electronics assembly includes at least one sensor and a controller having at least two joint-specific modules. Each joint-specific module is respectively configured to collect kinematic data resulting from movement of a respective specific joint sensed by the at least one sensor.

In another aspect, a universal payload includes an electronics assembly comprising at least one sensor and a housing enclosing the electronics assembly. The universal payload has a payload form factor that is configured to be at least partially inserted into a payload receptacle in each of the plurality of different orthopedic implants.

In another aspect, a universal payload includes a housing, at least one sensor aligned within the housing relative to an axis of a coordinate system of the universal payload to sense movement relative to the axis, and a payload alignment feature. The universal payload has a payload form factor that is configured to be at least partially inserted into a payload receptacle of each of a plurality of different orthopedic implants. The payload alignment feature is configured to engage an implant alignment feature of each of the plurality of different orthopedic implants to thereby align the coordinate system of the universal payload with a corresponding coordinate system of an orthopedic implant during insertion of the universal payload into the payload receptacle. Optionally, the payload alignment feature may comprise a tab that protrudes from a surface of the universal payload, and the implant alignment feature may comprise a slot that is recessed from a surface of the implant, where upon engagement of the universal payload with the implant, the tab fits into and engages with the slot. Optionally, the implant alignment feature may comprise a tab that protrudes from a surface of the implant, and the universal payload alignment feature may comprise a slot that is recessed from a surface of the universal payload, where upon engagement of the universal payload with the implant, the tab of the implant alignment features fits into and engages with the slot of the universal payload alignment feature. The slot and tab may be complementary in shape so that the tab fits snugly into the slot.

The present disclosure also relates to orthopedic implants that include an implant component configured to extend at least partially into a bone of a body and a payload receptacle in the implant component. The payload receptacle is configured to receive a universal payload that each of a plurality of different implant components is also configured to receive. In one aspect, the universal payload has an electronics assembly comprising at least one sensor, and a controller having at least two joint-specific modules, where each joint-specific module is respectively configured to collect kinematic data resulting from movement of a respective specific joint, as sensed by the at least one sensor.

In another aspect, a payload receptacle of the orthopedic implant has a receptacle form factor, and the orthopedic implant includes an implant alignment feature. The receptacle form factor is configured to receive a universal payload having a housing, at least one sensor aligned within the housing relative to an axis of a coordinate system of the universal payload to sense movement relative to the axis, and a payload alignment feature. The implant alignment feature is configured to engage the payload alignment feature to thereby align the coordinate system of the universal payload with a corresponding coordinate system of the orthopedic implant during insertion of the universal payload into the payload receptacle.

The present disclosure also relates to intelligent implants that include an orthopedic implant and a universal payload. The orthopedic implant has a payload receptacle configured to receive a component that each of a plurality of different orthopedic implants is configured to receive. The component may be a universal payload having a payload form factor that is configured to be at least partially inserted into the payload receptacle. In one aspect, the universal payload includes an electronics assembly and a housing enclosing the electronics assembly. The electronics assembly includes at least one sensor, and a controller having at least two joint-specific modules, where each joint-specific module is respectively configured to collect kinematic data resulting from movement of a respective specific joint, as sensed by the at least one sensor.

In another aspect, the orthopedic implant of the intelligent implant includes an implant alignment feature and a payload receptacle having a receptacle form factor. The universal payload includes at least one sensor aligned within the housing relative to an axis of a coordinate system of the universal payload to sense movement relative to the axis, and a payload alignment feature. The payload alignment feature is configured to engage the implant alignment feature to thereby align the coordinate system of the universal payload with a corresponding coordinate system of the orthopedic implant during insertion of the universal payload into the payload receptacle.

This Summary has been provided to introduce certain concepts in a simplified form that are further described in detail below in the Detailed Description. Except where otherwise expressly stated, this Summary is not intended to identify key or essential features of the claimed subject matter, nor is it intended to limit the scope of the claimed subject matter.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIGS. 1A and 1B are side-view and top-view illustrations of an embodiment of a universal payload.

FIG. 1C is an exploded view of the universal payload of FIG. 1A.

FIGS. 2A and 2B are detailed illustrations of an embodiment of a securing feature and an alignment feature of the universal payload.

FIG. 3 is an illustration of an orthopedic implant component of a hip arthroscopy system.

FIGS. 4A, 4B, and 4C are illustrations of a universal payload at various stages of integration into the hip implant component of FIG. 3.

FIG. 4D is a cross-section illustration of the securing feature of a universal payload coupled with a corresponding securing feature of the hip implant component of FIG. 3.

FIGS. 5A and 5B are illustrations of an orthopedic implant component of a knee arthroscopy system.

FIGS. 6A and 6B are illustrations of a universal payload at various stages of integration into the knee implant component of FIGS. 5A and 5B.

FIG. 6C is a phantom illustration of the securing feature of a universal payload coupled with a corresponding securing feature of the knee implant component of FIG. 6A.

FIGS. 7A, 7B, and 7C are illustrations of a universal payload integrated into an orthopedic implant component of a shoulder arthroscopy system.

FIG. 7D is a cross-section illustration of the securing feature and the alignment feature of a universal payload coupled with a corresponding securing feature and alignment feature of a shoulder implant component.

FIGS. 8A and 8B are respectively, an exploded perspective illustration and a cross-section illustration of an approach for integration of a universal payload with a host implant.

FIGS. 9A, 9B, and 9C are respectively, an exploded perspective illustration, a cross-section illustration, and a detailed illustration of another approach for integration of a universal payload with a host implant.

FIGS. 10A, 1013, and 10C are respectively, an exploded perspective illustration, a cross-section illustration, and a detailed illustration of another approach for integration of a universal payload with a host implant.

FIGS. 11A, 11B, and 11C are respectively, an exploded perspective illustration, a cross-section illustration, and a detailed illustration of another approach for integration of a universal payload with a host implant.

FIGS. 12A, 12B, and 12C are respectively, an exploded perspective illustration, a cross-section illustration, and a detailed illustration of another approach for integration of a universal payload with a host implant.

FIGS. 13A, 13B, and 13C are respectively, an exploded perspective illustration, a cross-section illustration, and a detailed illustration of another approach for integration of a universal payload with a host implant.

FIGS. 14A and 14B are respectively, an exploded perspective illustration and a cross-section illustration of another approach for integration of a universal payload with a host implant.

FIGS. 15A, 15B, and 15C are respectively, an exploded perspective illustration, a cross-section illustration, and a detailed illustration of another approach for integration of a universal payload with a host implant.

FIGS. 16A and 16B are respectively, a perspective illustration and a cross-section illustration of another approach for integration of a universal payload with a host implant.

FIGS. 17A 17B, 17C, 17D, and 17E are respectively, an exploded perspective illustration, a first cross-section illustration, a first detailed illustration, a second cross-section illustration, and a second detailed illustration of another approach for integration of a universal payload with a host implant.

FIGS. 18A, 18B, and 18C are respectively, an exploded perspective, illustration, a cross-section illustration, and a detailed illustration of another approach for integration of a universal payload with a host implant.

FIGS. 19A, 19B, and 19C are respectively, an exploded perspective illustration, a cross-section illustration, and a detailed illustration of another approach for integration of a universal payload with a host implant.

FIG. 20A is a block diagram of the electronics of a universal payload.

FIG. 20B is a perspective view of an inertial measurement unit (IMU) of the electronics of the universal payload FIG. 22 and of a set of coordinate axes within the frame of reference of the IMU.

FIG. 20C is an illustration of a set of coordinate axes of an IMU of a universal payload relative to a patient in which a host knee implant is implanted.

FIG. 20D is an illustration of a set of coordinate axes of an IMU of a universal payload relative to a patient in which a host shoulder implant is implanted.

FIG. 20E is an illustration of a set of coordinate axes of an IMU of a universal payload relative to a patient in which a host hip implant is implanted.

FIG. 21 is a context diagram of an intelligent implant, e.g., an orthopedic implant with an integrated universal payload, in a patient's home.

FIG. 22 is a flowchart of a method of implanting an intelligent implant, e.g., an orthopedic implant with a universal payload.

FIG. 23 is a flowchart of a method of manufacturing different orthopedic implants that are each configured to be integrated with the same universal payload.

FIG. 24 is an exploded perspective view of an embodiment of an alignment feature of the universal payload in engagement with an alignment feature of the implant.

### DETAILED DESCRIPTION

The present disclosure may be understood more readily by reference to the following detailed description of preferred embodiments of the disclosure and the examples of universal payloads configured for integration with each of a number of different types of orthopedic implants. The following description, along with the accompanying drawings, sets forth certain specific details in order to provide a thorough understanding of various disclosed embodiments. However, one skilled in the relevant art will recognize that the disclosed embodiments may be practiced in various combinations, without one or more of these specific details, or with other methods, components, devices, etc.

An implantable medical device (IMD) as used in the present disclosure, may be an implantable or implanted medical device that replaces or functionally supplements a natural body part. For example, an implantable medical device may be an orthopedic implant or a spinal implant.

An orthopedic implant as used in the present disclosure, may be a component of an orthopedic implant system or prosthesis that replaces or functionally supplements a joint of a patient, e.g., a knee, shoulder, or hip joint, and allows the patient to have the same, or nearly the same, mobility as would have been afforded by a healthy joint. Examples of orthopedic implant systems or prostheses include a total knee arthroscopy (TKA) prosthesis or system, such as a total knee implant; a total shoulder arthroscopy (TSA) prosthesis or system, such as a total shoulder implant; and a total hip arthroscopy (THA) prosthesis or system, such as a total hip implant. A non-limiting and non-exhaustive list of orthopedic implants include: 1) components of a TKA system, such as a tibial plate (with a tibial stem), a femoral component, a patellar component, a tibial extension; 2) components of a THA system, such as a femoral component, an acetabular component; and 3) components of a TSA system, such as a humeral stem component, a humeral stem adapter, a humeral head, a humeral head adapter, and a glenoid cap component.

A spinal implant as used in the present disclosure, may be a component of spinal implant system or prosthesis that replaces or functionally supplements a spine of a patient. Examples spinal implants include pedicle screws, spinal fusion devices, spinal cages, artificial discs, spinal rods, and spinal plates.

A universal payload as used in the present disclosure, is a component that can be integrated with each of a number of different implantable medical devices and is configured to monitor and report the status and/or activities of the implantable medical device, and the patient in which the implantable medical device is implanted. The universal payload includes electronics that may be configured to perform one or more of the following exemplary actions in order to characterize the post-implantation status of the implanted medical device: identifying the implanted medical device, e.g., by recognizing one or more unique identification codes for the implanted medical device; detecting, sensing and/or measuring parameters, which may collectively be referred to as monitoring parameters, in order to collect operational, kinematic, or other data about the implanted medical device, wherein such data may optionally be collected as a function of time; storing the collected data within the implanted medical device; and communicating the collected data and/or the stored data by a wireless means from the implanted medical device to an external computing device.

An intelligent implant as used in the present disclosure, refers to an implantable medical device with a universal payload, and is interchangeably referred to a smart device. When the intelligent implant collects kinematic data (described below), it may be referred to as a kinematic implantable device.

Kinematic data, as used herein, individually, or collectively includes some or all data associated with a particular kinematic implantable device and available for communication outside of the particular kinematic implantable device. For example, kinematic data may include raw data from one or more sensors of a kinematic implantable device, wherein the one or more sensors include such as gyroscopes, accelerometers, pedometers, strain gauges, and the like that produce data associated with motion, force, tension, velocity, or other mechanical forces. Kinematic data may also include processed data from one or more sensors, status data, operational data, control data, fault data, time data, scheduled data, event data, log data, and the like associated with the particular kinematic implantable device. In some cases, high resolution kinematic data includes kinematic data from one, many, or all of the sensors of the kinematic implantable device that is collected in higher quantities, resolution, from more sensors, more frequently, or the like.

In one embodiment, kinematics refers to the measurement of the positions, angles, velocities, and accelerations of body segments and joints during motion. Body segments are considered to be rigid bodies for the purposes of describing the motion of the body. They include the foot, shank (leg), thigh, pelvis, thorax, hand, forearm, upper-arm, and head. Joints between adjacent segments include the ankle (talocrural plus subtalar joints), knee, hip, wrist, elbow, and shoulder. Position describes the location of a body segment or joint in space, measured in terms of distance, e.g., in meters. A related measurement called displacement refers to the position with respect to a starting position. In two dimensions, the position is given in Cartesian co-ordinates, with horizontal followed by vertical position. In one embodiment, a kinematic implant or intelligent kinematic implants obtains kinematic data, and optionally only obtains kinematic data.

A sensor refers to a device that can be utilized to do one or more of detect, measure and/or monitor one or more different aspects of a body (anatomy, physiology, metabolism, movement, and/or function) and/or one or more aspects of an orthopedic device or implant (e.g., alignment in the patient, displacement or loosening in the patient). Representative examples of sensors suitable for use within the present disclosure include, for example, chemistry sensors (e.g., for blood and/or other fluids), metabolic sensors (e.g., for blood and/or other fluids), analyte sensors (e.g., for glucose), pH sensors, temperature sensors, fluid pressure sensors, fluid volume sensors, contact sensors, position sensors, pulse pressure sensors, blood volume sensors, blood flow sensors, accelerometers, gyroscopes, step counters, and mechanical stress sensors. Within certain embodiments the sensor can be a wireless sensor, or, within other embodiments, a sensor connected to a wireless microprocessor. Within further embodiments one or more (including all) of the sensors can have a Unique Sensor Identification number (“USI”) which specifically identifies the sensor.

### Universal Payload

The present disclosure provides a universal payload or cartridge that collects data related to a body part. The universal payload is configured to be integrated with each of various different types of orthopedic implants, referred to herein at times as “host implants”. For example, for the purpose of collecting data relevant to a knee, the universal payload may be integrated with a femoral component of a TKA system or a tibial component of a TKA system. For the purpose of collecting data relevant to the hip, the universal payload may be integrated with a femoral component of a THA system. For purposes of collecting data relevant to the shoulder, the universal payload may be integrated with a humeral component of a TSA system.

The integration of the universal payload into anyone of these host implants is enabled by a payload receptacle of the host implant. The payload receptacle is an empty space, a cavity, or a void in a rigid feature of the host implant. The empty space has a form factor configured to receive a universal payload. Form factor as used herein refers to physical characteristics, e.g., three-dimensional shape and size, of a structure or empty space.

The universal payload includes various electronics including one or more sensors and multiple joint-specific modules, each of which is configured to control data-collection functionality of the universal payload to collect data relevant to a particular joint of the body. For example, the joint-specific modules may include at least two of a knee module, a hip module, and a shoulder module. Each of these modules comprises firmware and/software configured to operate the universal payload in a manner specific to the host implant in which the universal payload is integrated.

The knee module is configured to control the data-collection functionality of the universal payload to obtain kinematic information associated with motion of a host knee implant. The knee module includes two submodules, one that operates the universal payload to obtain kinematic information associated with motion of a left knee implant, and a second that operate the universal payload to obtain kinematic information associated with motion of a right knee implant.

The hip module is configured to control the data-collection functionality of the universal payload to obtain kinematic information associated with motion of a host hip implant. The hip module includes two submodules, one that operates the universal payload to obtain kinematic information associated with motion of a left hip implant, and a second that operates the universal payload to obtain kinematic information associated with motion of a right hip implant.

The shoulder module is configured to control the data-collection functionality of the universal payload to obtain kinematic information associated with motion of a host shoulder implant. The shoulder module includes two submodules, one that operates the universal payload to obtain kinematic information associated with motion of a left shoulder implant, and a second that operates the universal payload to obtain kinematic information associated with motion of a right shoulder implant.

The universal payload also includes communication components, e.g., a transceiver and antenna, that transmits collected data and receives operational data, e.g., software/firmware updates.

### Universal Payload Structure

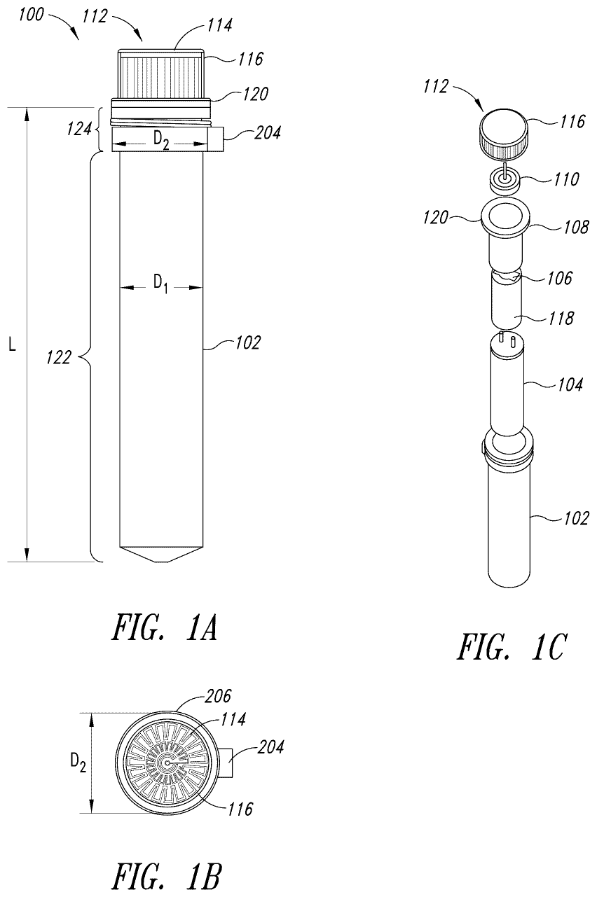

With reference to FIGS. 1A, 1B, and 1C, in some embodiments a universal payload 100 includes an outer casing or housing 102 that encloses a power component, e.g., a battery 104, an electronics assembly 106, a shroud 108, and a feedthrough assembly 110. The universal payload 100 further includes an antenna assembly 112 coupled to the housing 102. The antenna assembly 112 includes an antenna 114 that couples to the electronics assembly 106 through the feedthrough assembly 110, and a cover or cap 116 (transparent in FIGS. 1A, 1B, and 1C) that covers and protects the antenna 114. The cap 116 can be made from any material, such as plastic or ceramic, which allows radio-frequency (RF) signals to propagate through the radome with acceptable levels of attenuation and other signal degradation. In some embodiments the cap 116 is comprised of polyetherether-ketone (PEEK).

The housing 102 of the universal payload 100 includes a lower portion 122 and an upper portion 124. These portions 122, 124 are respectively sized and shaped to define a three-dimensional form factor of the universal payload 100 that enables the payload to fit into a payload receptacle of each of a plurality of different components of different orthopedic implants, where the payload receptacle has a form factor that receives the universal payload 100. The universal payload 100 shown in FIGS. 1A, 1B, and 1C has a cylindrical form factor. However, as described later in this disclosure, the form factor of the universal payload may have different geometries.

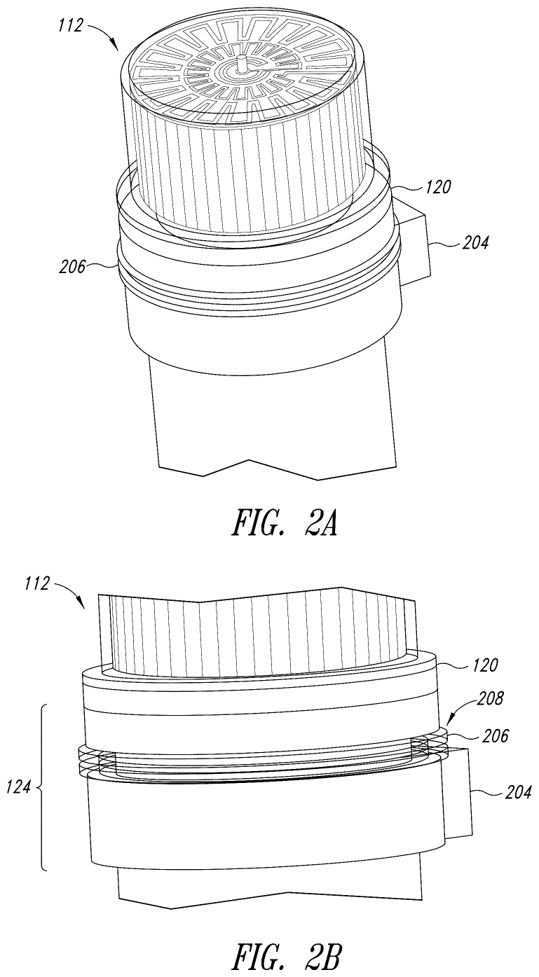

With continued reference to FIGS. 2A and 2B, in some embodiments, the housing 102 may have a length L between 42 mm and 44 mm. The overall length of the universal payload 100, from the bottom of the housing to the top of the antenna assembly 112 may be between 45 mm and 52 mm. Thus, the antenna assembly 112 may have a height between 3 mm and 8 mm. In some embodiments, the part of the housing 102 corresponding to the lower portion 122 of the universal payload 100 may have a lower diameter D₁ between 7.5 mm and 8.0 mm, while the part of the housing corresponding to the upper portion 124 of the universal payload may have an upper diameter D2 between 9.0 mm and 9.5 mm.

The upper portion 124 of the housing 102 includes an alignment feature 204, e.g., a tab, and a securing mechanism 206. As described later, the alignment feature 204 is configured to align with corresponding alignment features of different orthopedic implants or host implants. As also described later with reference to different orthopedic implants, aligning the alignment feature 204 with the corresponding alignment feature of a host implant ensures that one or more sensors in the universal payload 100 are in desired orientations relative to the host implant.

With continued reference to FIGS. 2A and 2B, the upper portion 124 of the housing 102 includes a securing mechanism 206. As described later, the securing mechanism 206 is configured to engage a corresponding securing feature of different orthopedic implants or host implants. In some embodiments, the securing mechanism 206 includes a coiled structure that is positioned in an annular groove 208 in the upper portion 124 of the housing 102. The coiled-structure securing mechanism 206 is configured to transition between a normally expanded state (as shown in FIGS. 2A and 2B) in which it has a first diameter in the range of 9.5 mm and 10.0 mm, and a compressed state in which it has a reduced diameter. As described later, the securing mechanism 206 is configured to engage the corresponding securing feature of different orthopedic implants or host implants.

With reference to FIGS. 1A, 1B, and 1C the battery 104 is configured to power the circuitry of the electronics assembly 106 of the universal payload 100 over a significant portion (e.g., 1-15+ years, e.g., 10 years, or 15 years), or the entirety (e.g., 18+ years), of the anticipated lifetime of the universal payload 100. In some embodiments, the battery 104 has a lithium-carbon-monofluoride (LiCFx) chemistry. With its LiCFx chemistry, the battery 104 can provide, over its lifetime, about 360 milliampere-hours (mAh) at 3.7 volts (V), although one can increase this output by about 36 mAh for each 5 mm of length added to the battery (similarly, one can decrease this output by about 36 mAh for each 5 mm of length subtracted from the battery). It is understood that other battery chemistries can be used if they can achieve the appropriate power requirements for a given application subject to the size and longevity requirements of the application. Some additional potential battery chemistries include, but are not limited to, Lithium ion (Li-ion), Lithium Manganese dioxide (Li—MnO2), silver vanadium oxide (SVO), Lithium Thionyl Chloride (Li—SOCl2), Lithium iodine, and hybrid types consisting of combinations of the above chemistries such as CFx-SVO.

The antenna assembly 112 includes an antenna 114 that couples to a radio transmitter integrated circuit of the electronics assembly 106 and a cap 116 that protects the antenna. The antenna 114 illustrated in this disclosure is a substrate antenna. In other configurations, the antenna 114 may be a loop antenna or a helix antenna. Examples of these antenna 114 configurations are disclosed, for example, in International PCT Application Serial No. PCT/US23/15224, which is incorporated by reference.

The electronics assembly 106 includes printed circuit boards surrounded by a liner 118. The liner 118 functions to mechanically stabilize the electronics assembly 106 within the housing 102. The electronics assembly 106 fits within and is surrounded by the shroud 108, which is formed of a biocompatible metallic material. In some embodiments, the material is titanium. The shroud 108 includes a flange 120.

The printed circuit boards of the electronics assembly 106 include one or more sensors and a controller configured to receive and process information from the one or more sensors relating to the state and functioning of the host implant in which the universal payload 100 is integrated, and the state of the patient within which the host implant is implanted. The electronics assembly 106 is further configured to transmit the processed information to an external device through the antenna assembly 112.

The electronics assembly 106 is coupled physically and electrically to the antenna 114 through terminals on the antenna terminal board, and to the battery 104 through terminals on the battery terminal board. The electronics assembly 106 may include an Inertial Measurement Unit (IMU) integrated circuit, a Real-Time Clock (RTC) integrated circuit, a memory integrated circuit (Flash), and other circuit components on one side, and a microcontroller (MCU) integrated circuit, a radio transmitter (RADIO) integrated circuit, and other circuit components on the other side. In any event, the folded electronics assembly 106 provides a compact configuration that conserves a significant amount of physical space in the universal payload. Details on the electronics assembly 106 are provided below. Additional details are also disclosed in PCT Publication No. WO 2022/266085, which is incorporated by reference.

### Universal Payload and Host Implant Integration

Generally speaking, disclosed herein are intelligent implants comprising a universal payload and a host implant having a payload receptacle. The universal payload and the payload receptacle are configured such that the universal payload can be integrated with the host implant, where such integration is achieved through respective configurations of the universal payload and the host implant that enable insertion of the universal payload into the payload receptacle and securing of the payload therein. To this end, in the embodiments disclosed herein, the universal payload and the host implant have corresponding securing features that together function to secure the universal payload within the host implant upon insertion of the universal payload into the payload receptacle, where upon insertion includes the time when insertion is complete, and the universal payload has been finally inserted into the payload receptacle. It may be said that the two corresponding securing features are complementary to one another so that when the two features are engaged or joined together, they interact to create a connection that securely holds the universal payload within the payload receptacle. A securing feature may also be referred to as a locking mechanism or a securing mechanism.

In some embodiments, the securing features provide a force-fit or friction-fit that secures the universal payload in the host implant independent of any other securing features. In other words, the universal payload is secured within the payload receptacle solely by frictional forces. Particularly when securing a universal payload in a payload receptacle solely by friction forces, pressure may be used to force the two complementary pieces together, where pressure develops a so-called force-fit between the pieces, whereby the pieces are held together by frictional forces. In some embodiments, the securing features secure the universal payload in the host implant independent of any friction force. Particularly, the universal payload comprises a feature configured solely as a securing feature, and the payload receptacle comprises a feature configured solely as a securing feature that is complementary to the securing feature of the universal payload.

In some embodiments, the universal payload is aligned relative to a host implant and is secured within a payload receptacle of the host implant to achieve at least two results. A first result is that the universal payload cannot easily be removed from the host implant. The universal payload is secured within the payload receptacle so that after the host implant is implanted in a patient, the universal payload remains secured during normal daily activities of the patient for the expected lifetime of the payload. The second result is that the universal payload remains in constant alignment with the host implant. The universal payload is secured within the payload receptacle so that after the host implant is implanted in the patient, the universal payload does not rotate within the payload receptacle during normal daily activities of the patient for the expected lifetime of the payload.

Following are examples of intelligent implants, wherein a same universal payload can be integrated with each of a different type or configuration of an orthopedic implant, including a hip implant, a knee implant, and a shoulder implants.

### Hip Implant Integration

With reference to FIG. 3, an orthopedic implant component 300 of a hip arthroscopy system includes a payload receptacle 302 having a form factor configured to receive a universal payload 100. The payload receptacle 302 may be formed by drilling into a solid portion of the implant component 300 or the implant component may be formed to include the payload receptacle. The payload receptacle 302 includes a lower portion 304 having length and diameter dimensions sized to receive the lower portion 122 of the housing 102 of a universal payload 100. The payload receptacle 302 also includes an upper region 306 having length and diameter dimensions sized to receive the upper portion 124 of the housing 102 of a universal payload 100. The upper region 306 of the payload receptacle 302 includes an annular recess 308 corresponding to a securing feature. The annular recess 308 is positioned and configured to receive a corresponding securing mechanism 206, e.g., coiled structure, of the universal payload 100. The payload receptacle 302 also includes an alignment feature 310 configured to receive a corresponding alignment feature 204, e.g., tab, of the universal payload 100.

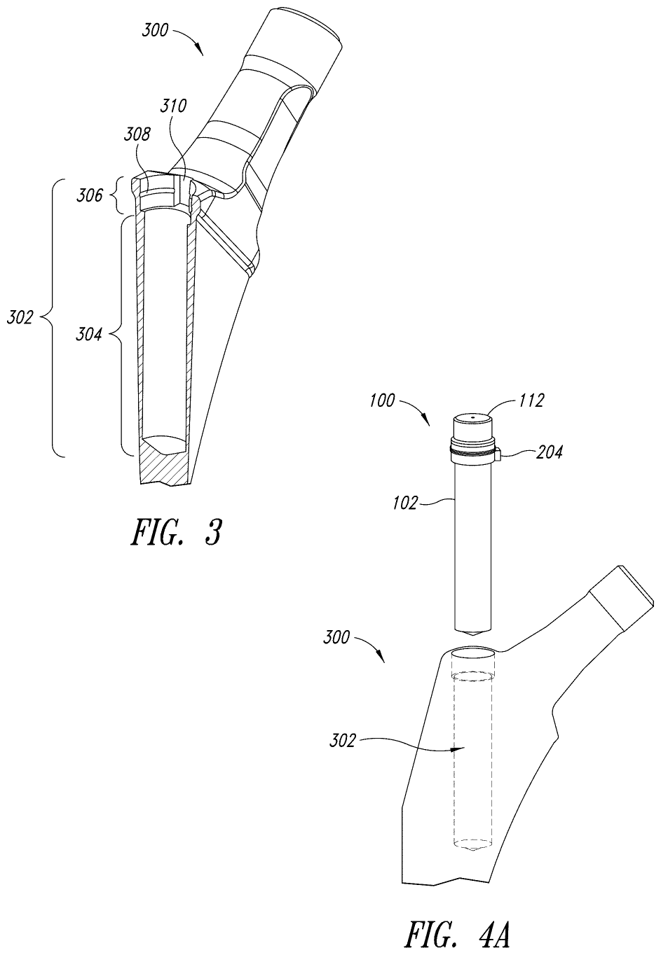

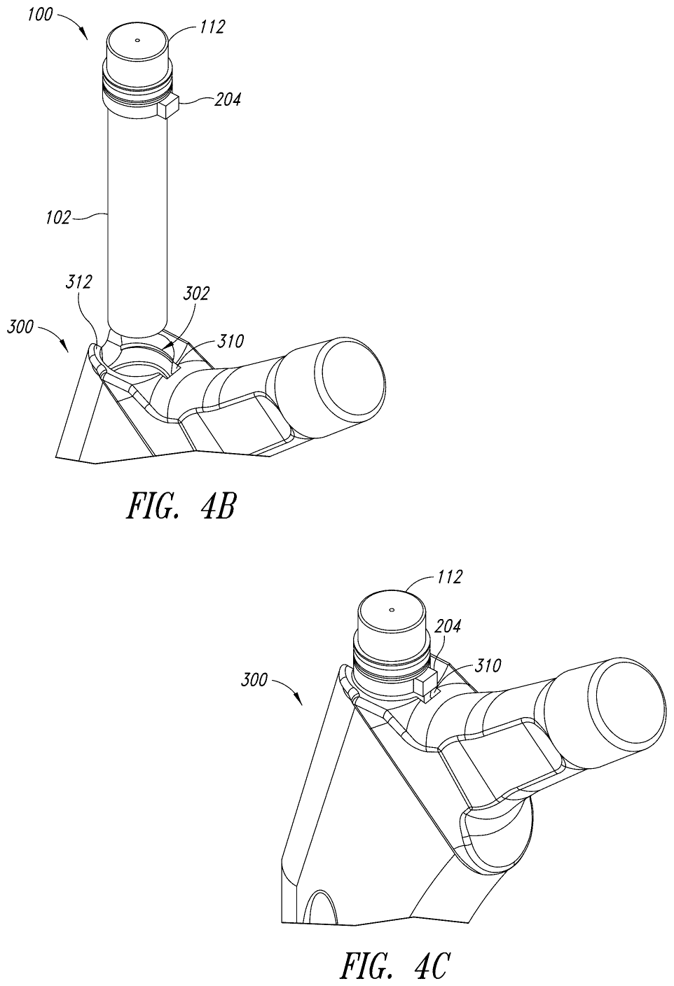

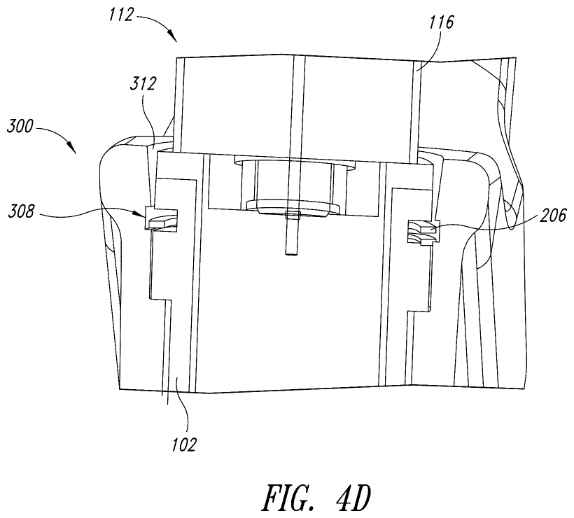

With reference to FIGS. 4A, 4B, 4C, and 4D, a universal payload 100 is integrated with the hip implant component 300 by aligning the housing 102 with the payload receptacle 302, including in particular, the respective alignment features 204, 310, and inserting the housing into the payload receptacle until the universal payload is fully seated in the payload receptacle. During insertion, at the point where the securing mechanism 206 of the universal payload 100 abuts the upper end of the payload receptacle 302, the securing mechanism is forced into its compressed state. Transitioning to a compressed state may be aided by a tapper 312 formed at the upper end of the payload receptacle 302. Upon continued insertion, the securing mechanism 206 eventually aligns with the annular recess 308 of the payload receptacle 302, at which point, the securing mechanism transitions to its normally expanded state to partially rest in the annular recess 308. In its normally expanded state, the securing mechanism 206 secures the universal payload 100 in place in the payload receptacle 302 and prevents removal of the payload. When the universal payload 100 is fully seated in the payload receptacle 302, the antenna assembly 112 of the payload is exposed and projects from a surface of the hip implant component.

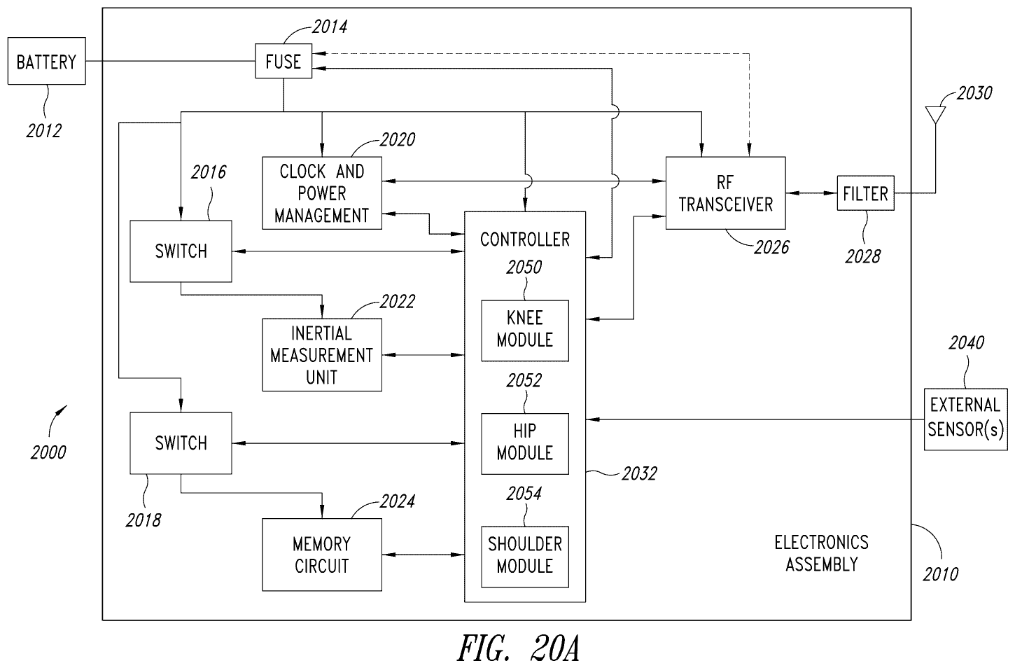

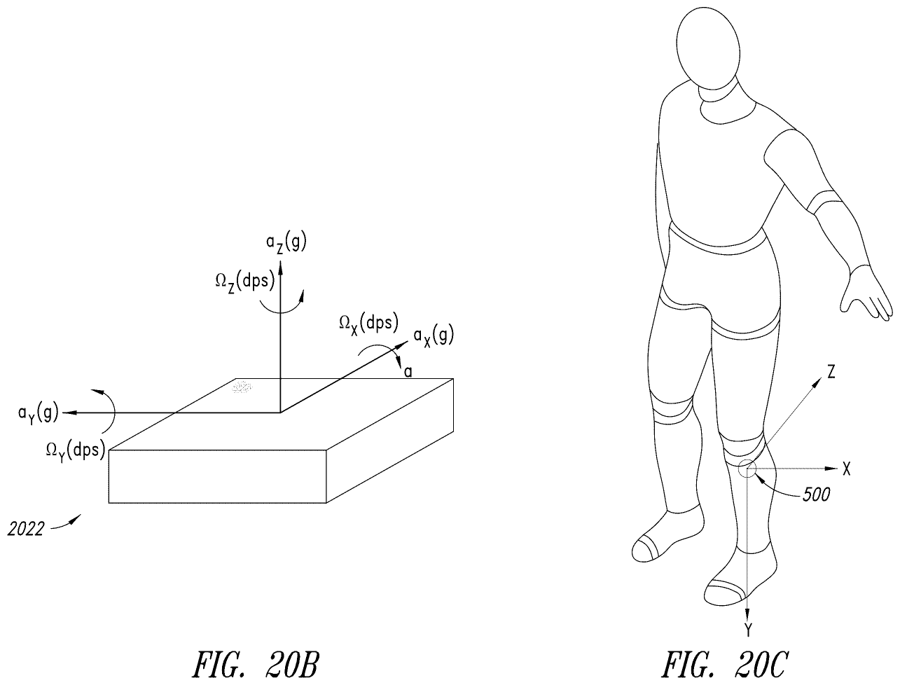

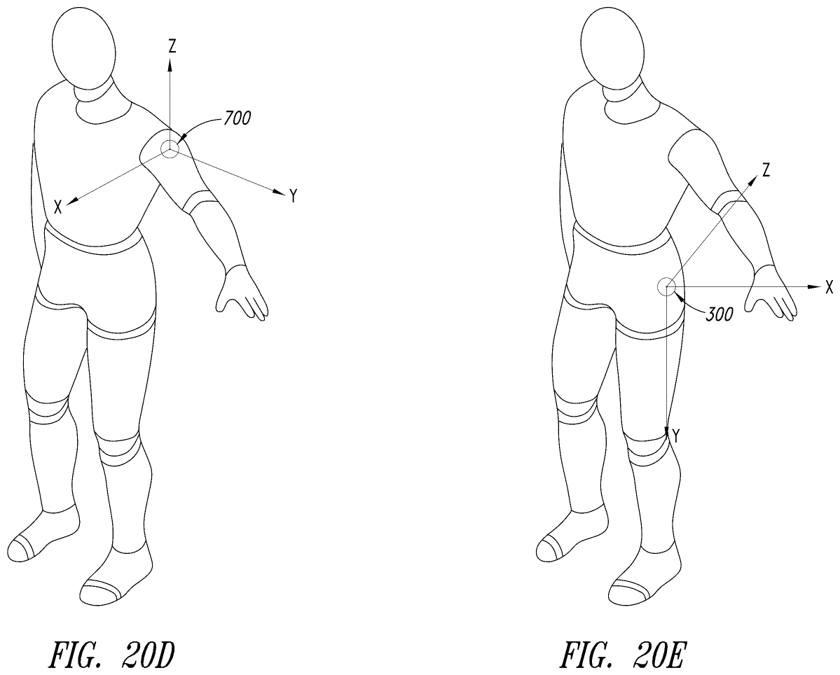

The alignment features 204, 310 of the universal payload 100 and the host hip implant 300 ensure that one or more sensors in the universal payload 100 are in a desired orientation relative to the host hip implant. For example, with reference to FIGS. 20A and 20B, the universal payload 100 may include an inertial measurement unit (IMU) 2022 that has a frame of reference with coordinate x, y, and z axes, and that can be configured to measure, or to otherwise quantify, acceleration that the IMU experiences along each of the x, y, and z axes, and angular velocity that the IMU experiences about each of the x, y, and z axes. With reference to FIG. 20E, the hip host implant 300 has a frame of reference with coordinate x, y, and z axes relative to the hip of the patient in which the host implant is implanted. For the IMU 2022 to collect meaningful data the respective x, y, z axes of the IMU coordinate system should be aligned with the x, y, z axes of the coordinate system of the host hip implant 300. The alignment features 204, 310 of the universal payload 100 and the host hip implant 300 ensure alignment of the respective axes within a degree of tolerance.

### Knee Implant Integration

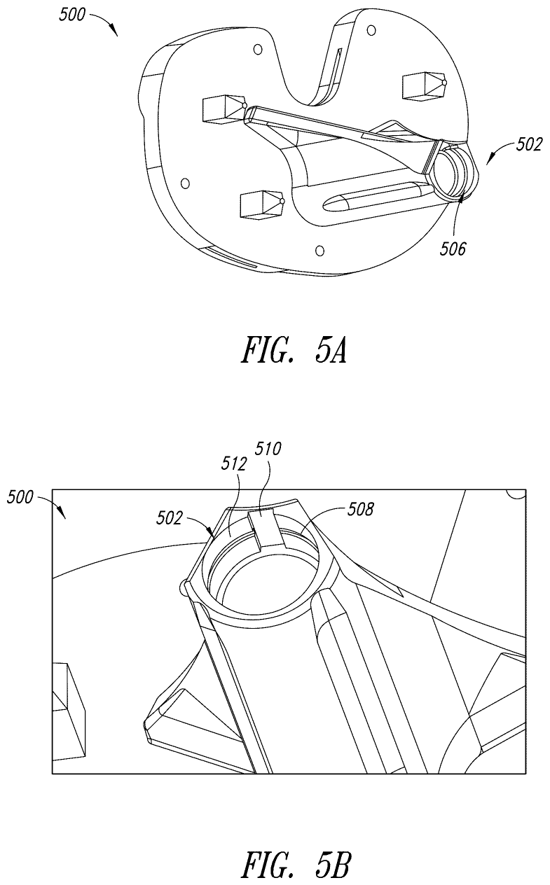

With reference to FIGS. 5A and 5B, an orthopedic implant component 500 of a knee arthroscopy system includes a payload receptacle 502 having a form factor configured to receive a universal payload 100. The payload receptacle 502 has the same form factor as the payload receptacle 302 of the hip implant component 300 of FIG. 3. Thus, the same universal payload 100 that can be implanted in the hip implant component 300 of FIG. 3 can be inserted in the knee implant component 500.

The payload receptacle 502 may be formed by drilling into a solid portion of the implant component 500 or the implant component may be formed to include the payload receptacle. Similar to the payload receptacle of the hip implant component, payload receptacle 502 of the knee implant component 500 includes a lower portion (not visible) having length and diameter dimensions sized to receive the lower portion 122 of the housing 102 of a universal payload 100. The payload receptacle 502 also includes an upper region 506 having length and diameter dimensions sized to receive the upper portion 124 of the housing 102 of a universal payload 100. The upper region 506 of the payload receptacle 502 includes an annular recess 508 corresponding to a securing feature. The annular recess 508 is positioned and configured to receive a corresponding securing mechanism 206, e.g., coiled structure, of the universal payload 100. The payload receptacle 502 also includes an alignment feature 510 configured to receive a corresponding alignment feature 204, e.g., tab, of the universal payload 100.

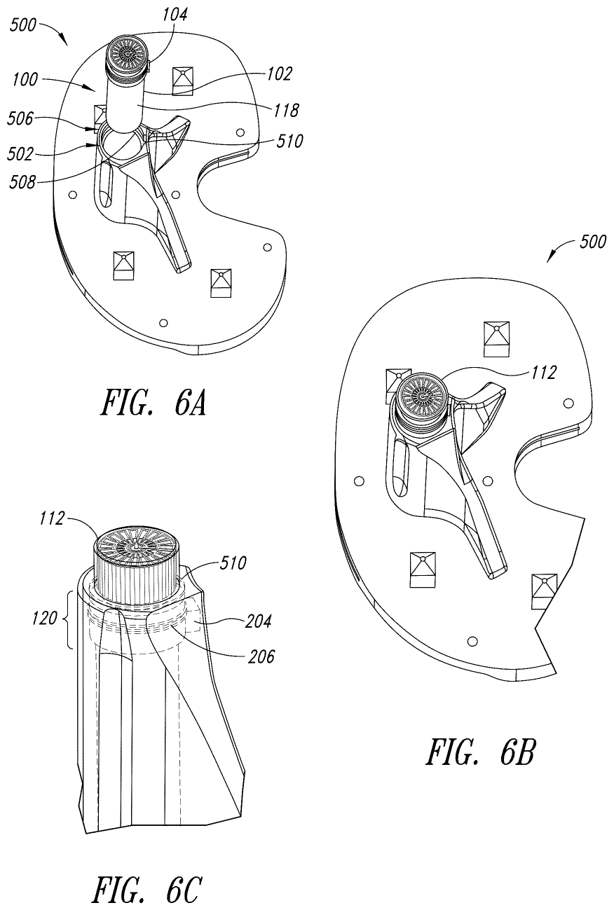

With reference to FIGS. 6A, 6B, and 6C, a universal payload 100 is integrated with the knee implant component 500 by aligning the housing 102 with the payload receptacle 502, including in particular, the respective alignment features 204, 510, and inserting the housing into the payload receptacle until the universal payload is fully seated in the payload receptacle. During insertion, at the point where the securing mechanism 206 of the universal payload 100 abuts the upper end of the payload receptacle 502, the securing mechanism is forced into its compressed state. Transitioning to a compressed state may be aided by a taper 512 (shown in FIG. 5B) formed at the upper end of the payload receptacle 502. Upon continued insertion, the securing mechanism 206 eventually aligns with the annular recess 508 of the payload receptacle, at which point, the securing mechanism transitions to its normally expanded state to partially rest in the annular recess 508. In its normally expanded state, the securing mechanism 206 secures the universal payload 100 in place in the payload receptacle 502 and prevents removal of the payload. When the universal payload 100 is fully seated in the payload receptacle 502, the antenna assembly 112 of the payload is exposed and projects from a surface of the hip implant component.

The alignment features 204, 510 of the universal payload 100 and the host knee implant 500 ensure that one or more sensors in the universal payload 100 are in a desired orientation relative to the host knee implant. For example, with reference to FIGS. 20A and 20B, the universal payload 100 may include an inertial measurement unit (IMU) 2022 that has a frame of reference with coordinate x, y, and z axes, and that can be configured to measure, or to otherwise quantify, acceleration that the IMU experiences along each of the x, y, and z axes, and angular velocity that the IMU experiences about each of the x, y, and z axes. With reference to FIG. 20C, the host knee implant 500 has a frame of reference with coordinate x, y, and z axes relative to the knee of the patient in which the host implant in implanted. For the IMU 2022 to collect meaningful data the respective x, y, z axes of the IMU coordinate system should be aligned with the x, y, z axes of the coordinate system of the host knee implant 500. The alignment features 204, 510 of the universal payload 100 and the host knee implant 500 ensure alignment of the respective axes within a degree of tolerance.

### Shoulder Implant Integration

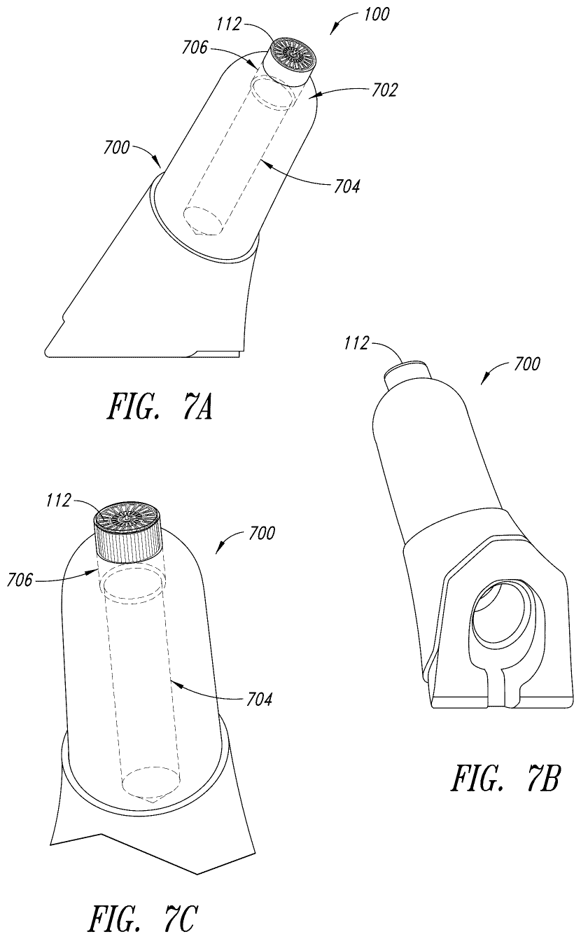

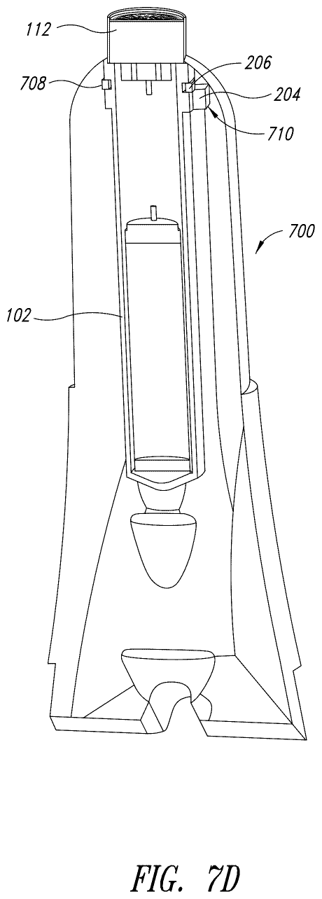

With reference to FIGS. 7A, 7B, 7C, and 7D, an orthopedic implant component 700 of a shoulder arthroscopy system includes a payload receptacle 702 having a form factor configured to receive a universal payload 100. The payload receptacle 702 has the same form factor as the payload receptacle 302 of the hip implant component 300 of FIG. 3 and the payload receptacle 502 of the knee implant component 500 of FIGS. 5A and 5B. Thus, the same universal payload 100 that can be implanted in the hip implant component 300 of FIG. 3 and the knee implant component 500 of FIGS. 5A and 5B can be inserted in the shoulder implant component 700.

The payload receptacle 702 may be formed by drilling into a solid portion of the implant component 700 or the implant component may be formed to include the payload receptacle. Similar to the payload receptacle of the hip implant component and the knee implant component, the payload receptacle 702 of the shoulder implant component 700 includes a lower portion 704 having length and diameter dimensions sized to receive the lower portion 122 of the housing 102 of a universal payload 100. The payload receptacle 702 also includes an upper region 706 having length and diameter dimensions sized to receive the upper portion 124 of the housing 102 of a universal payload 100. The upper region 706 of the payload receptacle 702 includes an annular recess 708 corresponding to a securing feature. The annular recess 708 is positioned and configured to receive a corresponding securing mechanism 206, e.g., coiled structure, of the universal payload 100. The payload receptacle 702 also includes an alignment feature 710 configured to receive a corresponding alignment feature 204, e.g., tab, of the universal payload 100.

A universal payload 100 is integrated with the shoulder implant component 700 by aligning the housing 102 with the payload receptacle 702, including in particular, the respective alignment features 204, 710, and inserting the housing into the payload receptacle until the universal payload is fully seated in the payload receptacle. During insertion, at the point where the securing mechanism 206 of the universal payload 100 abuts the upper end of the payload receptacle 702, the securing mechanism is forced into its compressed state. Transitioning to a compressed state may be aided by a tapper (not visible) formed at the upper end of the payload receptacle 702. Upon continued insertion, the securing mechanism 206 eventually aligns with the annular recess 708 of the payload receptacle, at which point, the securing mechanism transitions to its normally expanded state to partially rest in the annular recess 708. In its normally expanded state, the securing mechanism 206 secures the universal payload 100 in place in the payload receptacle 702 and prevents removal of the payload. When the universal payload 100 is fully seated in the payload receptacle 702, the antenna assembly 112 of the payload is exposed and projects from a surface of the hip implant component.

The alignment features 204, 710 of the universal payload 100 and the host shoulder implant 700 ensure that one or more sensors in the universal payload 100 are in a desired orientation relative to the host shoulder implant. For example, with reference to FIGS. 20A and 20B, the universal payload 100 may include an inertial measurement unit (IMU) 2022 that has a frame of reference with coordinate x, y, and z axes, and that can be configured to measure, or to otherwise quantify, acceleration that the IMU experiences along each of the x, y, and z axes, and angular velocity that the IMU experiences about each of the x, y, and z axes. With reference to FIG. 20D, the host shoulder implant 700 has a frame of reference with coordinate x, y, and z axes relative to the shoulder of the patient in which the host implant is implanted. For the IMU 2022 to collect meaningful data the respective x, y, z axes of the IMU coordinate system should be aligned with the x, y, z axes of the coordinate system of the host shoulder implant. The alignment features 204, 710 of the universal payload 100 and the host shoulder implant 700 ensure alignment of the respective axes within a degree of tolerance.

### Other Alignment and Securing Approaches

Respective portions of the universal payload and the host implant may have other configurations and features the enable alignment and/or securing of the universal payload during integration with a host implant. The following are several examples of other approaches to align and/or secure a universal payload in a host implant.

### Lateral Clip

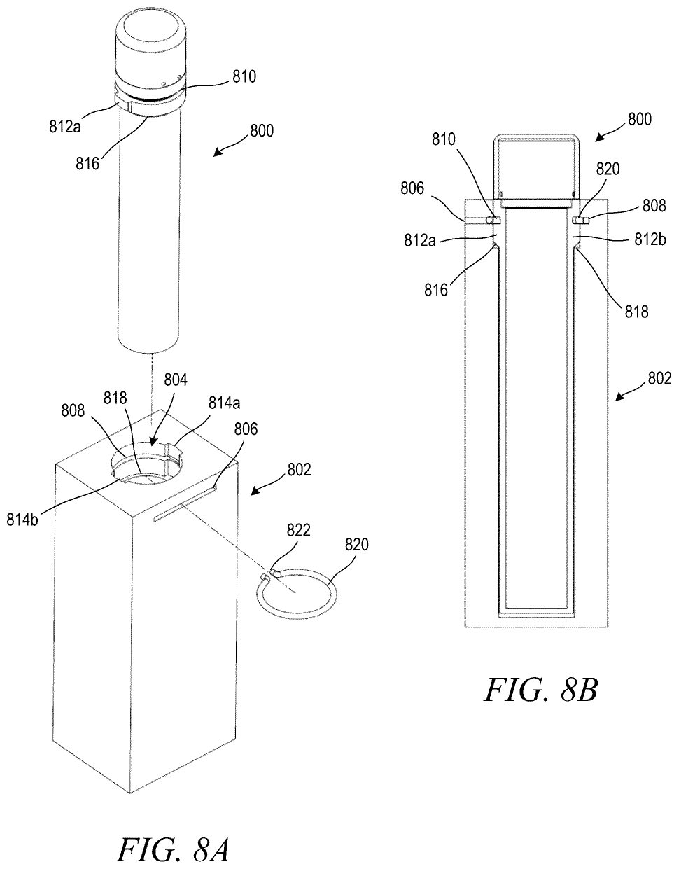

With reference to FIGS. 8A and 8B, integration of a universal payload 800 with a host implant 802 can be enabled by respective alignment features of the payload and implant, respective securing features of the payload and implant, and an additional securing element, e.g., a retaining clip 820. While the host implant 802 is generically illustrated as a block, the implant can be any one of the orthopedic implants described in the present disclosure.

In this embodiment, the host implant 802 includes a payload receptacle 804 or cavity bounded by an interior wall of the host implant. The payload receptacle 804 has a form factor configured to receive a universal payload 800. The host implant 802 also includes a slot 806 that extends through a thickness of the host implant to the payload receptacle 804. An annular groove 808 is formed in the interior wall of the host implant 802 opposite the slot 806 and is horizontally aligned with the slot. The upper region of the universal payload 800 includes an annular groove 810 and pair of alignment tabs 812a, 812b. The annular groove 810 is configured to receive a portion of a retaining clip 820.

During integration of the universal payload 800 with a host implant 802, the alignment tabs 812a, 812b of the universal payload 800 are aligned with corresponding alignment slots 814a, 814b of the host implant 802, and the universal payload 800 is inserted into the payload receptacle 804 until the alignment tabs 812a, 812b and a shoulder 816 of the payload rest on an annular ledge 818 of the payload receptacle 804. After the universal payload 800 is fully inserted into the payload receptacle 804, the annular groove 810 of the universal payload 800 is aligned with the annular groove 808 of the host implant 802 on one side and the slot 806 on the other side. A retaining clip 820 is inserted through the slot 806. As the tips 822 of the retaining clip 820 encounter the annular groove 810 of the universal payload 800, the ring flexes such that the space between the tips expands and the ring clasps around the payload. Upon complete insertion of the retaining clip 820, the ring rests partially within the annular groove 810 of the universal payload 800 and partially within the annular groove 808 of the host implant 802 to thereby secure the universal payload within the host implant.

Elongated Tab with Vertical Screw

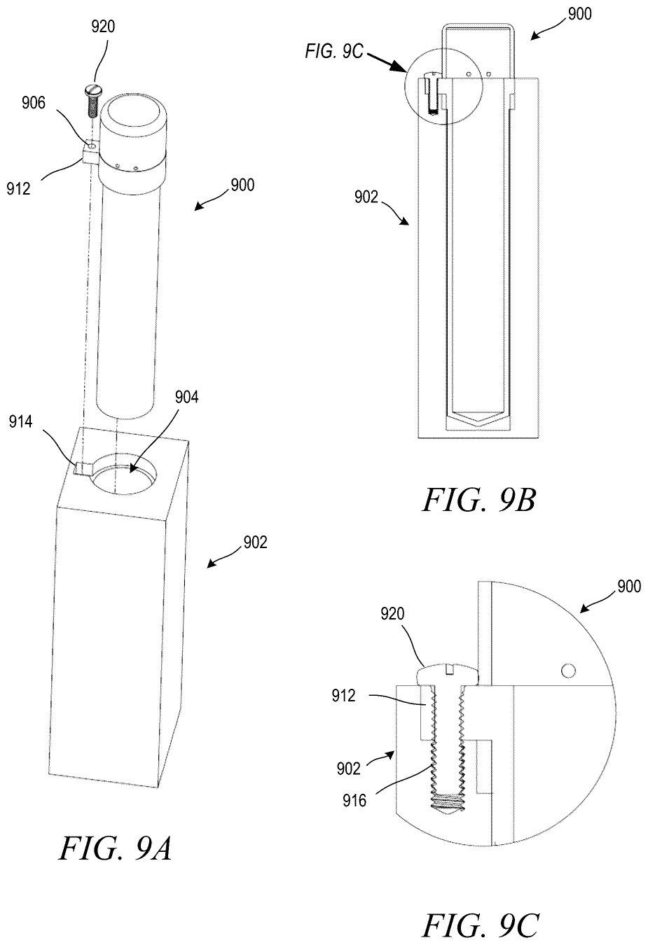

With reference to FIGS. 9A, 9B, and 9C, integration of a universal payload 900 with a host implant 902 can be enabled by respective alignment features of the payload and implant, respective securing features of the payload and implant, and an additional securing element, e.g., a retaining screw 920. While the host implant 902 is generically illustrated as a block, the implant can be any one of the orthopedic implants described in the present disclosure.

In this embodiment, the host implant 902 includes a payload receptacle 904 or cavity bounded by an interior wall of the host implant. The payload receptacle 904 has a form factor configured to receive a universal payload 900. The host implant 902 also includes an alignment slot 914 and a hole 916 at a bottom surface of the slot. The upper region of the universal payload 900 includes an alignment tab 912 having a through-hole 906.

During integration of the universal payload 900 with a host implant 902, the alignment tab 912 of the universal payload 900 is aligned with the alignment slot 914 of the host implant 902, and the universal payload 900 is inserted into the payload receptacle 904 until the bottom of the alignment tab 912 rests on the bottom surface of the alignment slot 914 of the payload receptacle 904. After the universal payload 900 is fully inserted into the payload receptacle 904, the through-hole 906 of the universal payload 900 is aligned with the hole 916 of the host implant 902. A retaining screw 920 is screwed through the through-hole 906 of the universal payload 900 into the hole 916 of the host implant 902 to thereby secure the universal payload 900 within the host implant.

Elongated Tabs with Vertical Screws

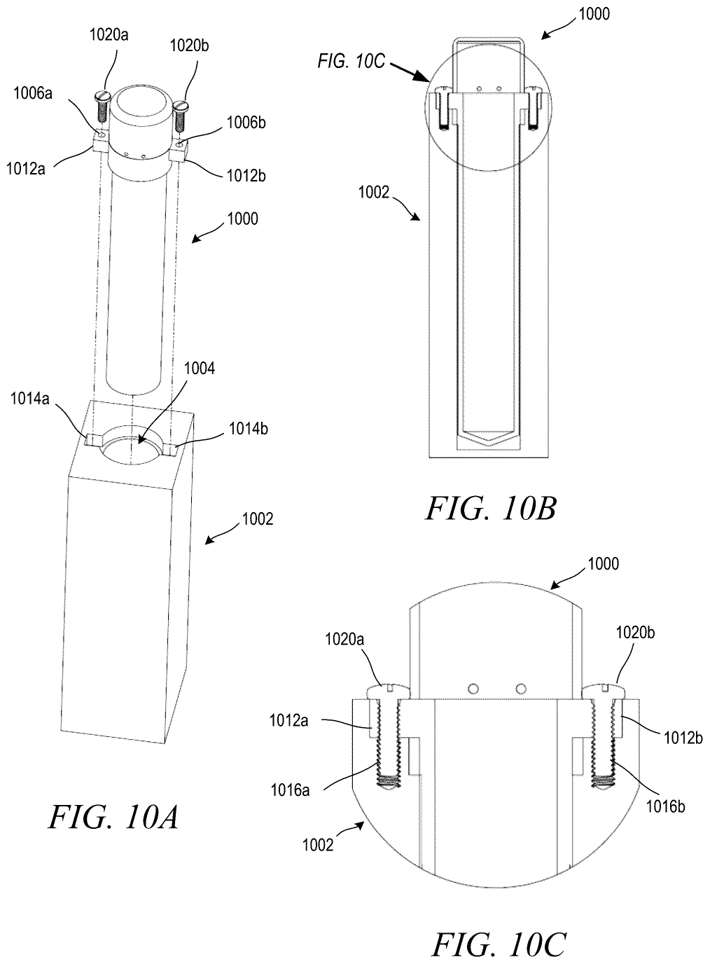

With reference to FIGS. 10A, 1013, and 10C, integration of a universal payload 1000 with a host implant 1002 can be enabled by respective alignment features of the payload and implant, respective securing features of the payload and implant, and an additional securing element, e.g., a pair of retaining screw 1020a, 1020b. While the host implant 1002 is generically illustrated as a block, the implant can be any one of the orthopedic implants described in the present disclosure.

In this embodiment, the host implant 1002 includes a payload receptacle 1004 or cavity bounded by an interior wall of the host implant. The payload receptacle 1004 has a form factor configured to receive a universal payload 1000. The host implant 1002 also includes a pair of alignment slots 1014a, 1014b, each with a hole 1016a, 1016b at a bottom surface of the slot. The upper region of the universal payload 1000 includes a pair of alignment tabs 1012a, 1012b, each with a through-hole 1006a, 1006b.

During integration of the universal payload 1000 with a host implant 1002, the alignment tabs 1012a, 1012b of the universal payload 1000 are aligned with the alignment slots 1014a, 1014b of the host implant 1002, and the universal payload 1000 is inserted into the payload receptacle 1004 until the bottom of the alignment tabs 1012a, 1012b rest on the bottom surfaces of the alignment slots 1014a, 1014b of the payload receptacle 1004. After the universal payload 1000 is fully inserted into the payload receptacle 1004, the through-holes 1006a, 1006b of the universal payload 1000 are aligned with the holes 1016a, 1016b of the host implant 1002. Retaining screws 1020a, 1020b are screwed through the through-holes 1006a, 1006b of the universal payload 1000 into the holes 1016a, 1016b of the host implant 1002 to thereby secure the universal payload 1000 within the host implant.

Elongated Tabs with Vertical Screws and Plate

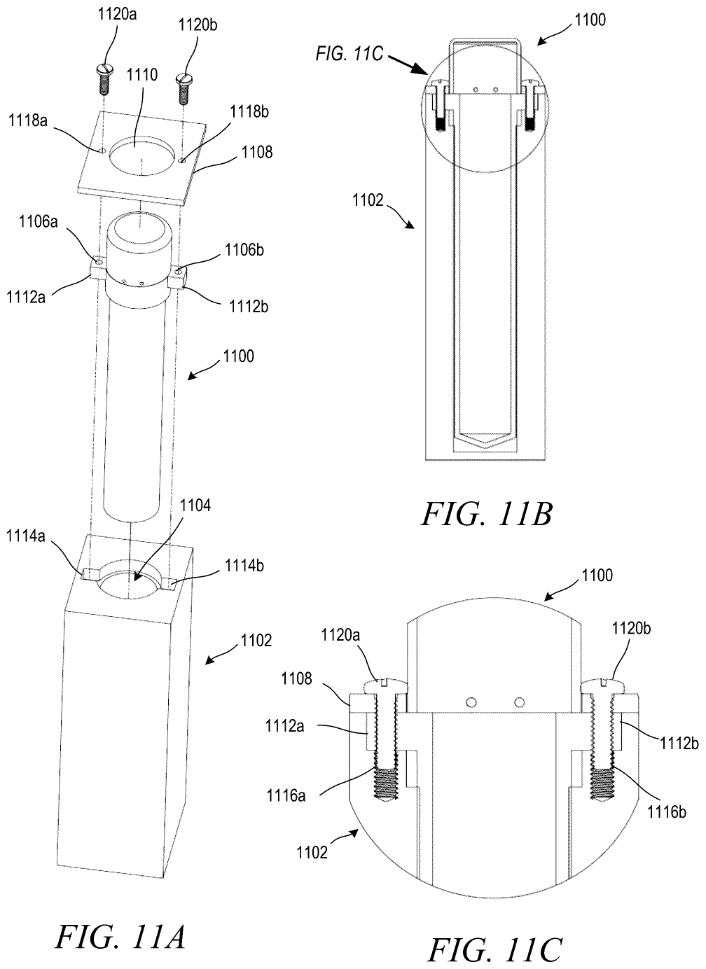

With reference to FIGS. 11A, 11B, and 11C, integration of a universal payload 1100 with a host implant 1102 can be enabled by respective alignment features of the payload and implant, respective securing features of the payload and implant, and additional securing elements, e.g., a pair of retaining screw 1120a, 1120b and a retaining plate 1108 with an antenna through-hole 1110 and a pair of through-holes 1118a, 1118b. While the host implant 1102 is generically illustrated as a block, the implant can be any one of the orthopedic implants described in the present disclosure.

In this embodiment, the host implant 1102 includes a payload receptacle 1104 or cavity bounded by an interior wall of the host implant. The payload receptacle 1104 has a form factor configured to receive a universal payload 1100. The host implant 1102 also includes a pair of alignment slots 1114a, 1114b, each with a hole 1116a, 1116b at a bottom surface of the slot. The upper region of the universal payload 1100 includes a pair of alignment tabs 1112a, 1112b, each with a through-hole 1106a, 1106b.

During integration of the universal payload 1100 with a host implant 1102, the alignment tabs 1112a, 1112b of the universal payload 1100 are aligned with the alignment slots 1114a, 1114b of the host implant 1102, and the universal payload 1100 is inserted into the payload receptacle 1104 until the bottom of the alignment tabs 1112a, 1112b rest on the bottom surfaces of the alignment slots 1114a, 1114b of the payload receptacle 1104. After the universal payload 1100 is fully inserted into the payload receptacle 1104, the through-holes 1106a, 1106b of the universal payload 1100 are aligned with the holes 1116a, 1116b of the host implant 1102. The retaining plate 1108 is placed on the host implant 1102 with the antenna assembly of the universal payload 1100 extending through the antenna through-hole 1110. Retaining screws 1120a, 1120b are screwed through the through-holes 1118a, 1118b of the plate 1108, through the through-holes 1106a, 1106b of the universal payload 1100, and into the holes 1116a, 1116b of the host implant 1102 to thereby secure the universal payload 1100 within the host implant.

### Horizontal Set Screw

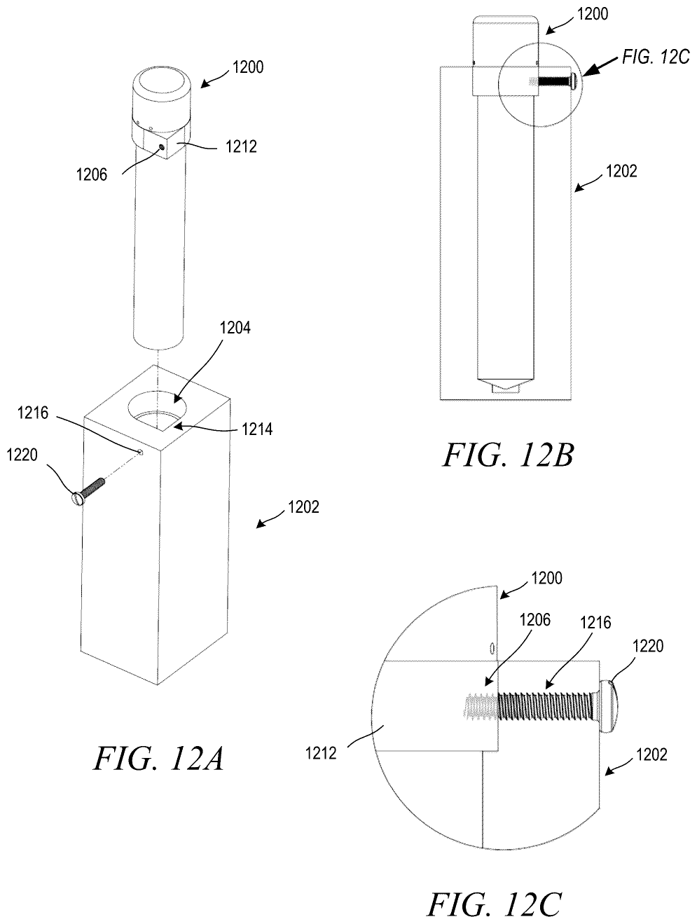

With reference to FIGS. 12A, 12B, and 12C, integration of a universal payload 1200 with a host implant 1202 can be enabled by respective alignment features of the payload and implant, respective securing features of the payload and implant, and an additional securing element, e.g., a retaining screw 1220. While the host implant 1202 is generically illustrated as a block, the implant can be any one of the orthopedic implants described in the present disclosure.

In this embodiment, the host implant 1202 includes a payload receptacle 1204 or cavity bounded by an interior wall of the host implant. The payload receptacle 1204 has a form factor configured to receive a universal payload 1200. The host implant 1202 also includes an alignment feature 1214 and a through-hole 1216 that extends through a thickness of the host implant. The upper region of the universal payload 1200 includes an alignment tab 1212 having a hole 1206 that extends into a side of the alignment tab.

During integration of the universal payload 1200 with a host implant 1202, the alignment tab 1212 of the universal payload is aligned with the alignment feature 1214 of the host implant, and the universal payload is inserted into the payload receptacle 1204 until the bottom of the alignment tab 1212 rests on the bottom surface of the alignment feature 1214 of the payload receptacle 1204. After the universal payload 1200 is fully inserted into the payload receptacle 1204, the hole 1206 of the universal payload 1200 is aligned with the through-hole 1216 of the host implant 1202. A retaining screw 1220 is screwed through the through-hole 1216 of the host implant 1202 into the hole 1206 of the universal payload 1200 to thereby secure the universal payload within the host implant.

### Horizontal Set Screw Over Tab

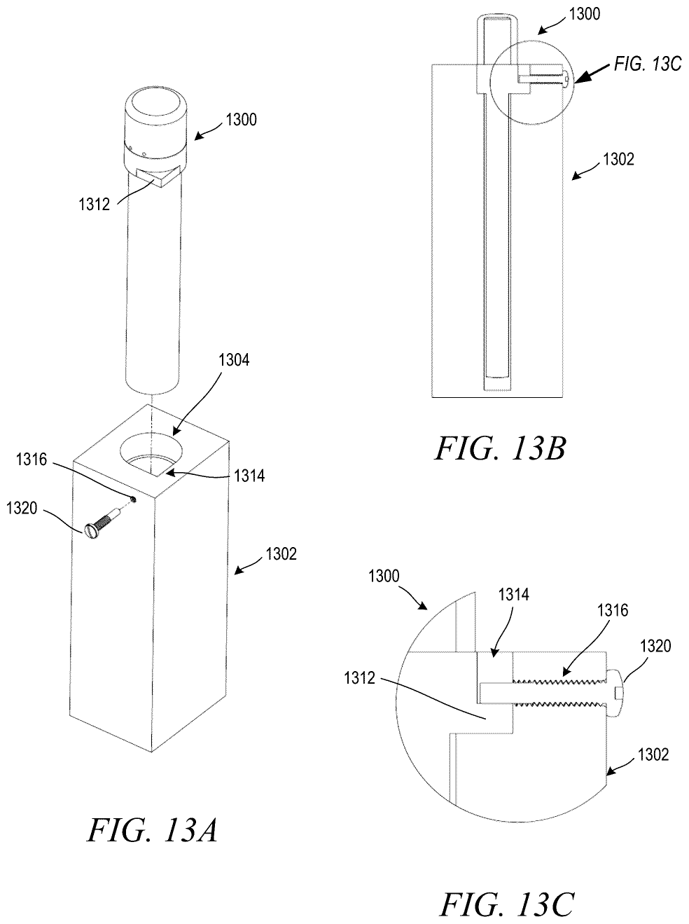

With reference to FIGS. 13A, 13B, and 13C, integration of a universal payload 1300 with a host implant 1302 can be enabled by respective alignment features of the payload and implant, respective securing features of the payload and implant, and an additional securing element, e.g., a retaining screw 1320. While the host implant 1202 is generically illustrated as a block, the implant can be any one of the orthopedic implants described in the present disclosure.

In this embodiment, the host implant 1302 includes a payload receptacle 1304 or cavity bounded by an interior wall of the host implant. The payload receptacle 1304 has a form factor configured to receive a universal payload 1300. The host implant 1302 also includes an alignment feature 1314 and a through-hole 1316 that extends through a thickness of the host implant. The upper region of the universal payload 1300 includes an alignment tab 1312 having a having a reduced thickness in comparison to the alignment tab 1212 of FIG. 12A.

During integration of the universal payload 1300 with a host implant 1302, the alignment tab 1312 of the universal payload is aligned with the alignment feature 1314 of the host implant, and the universal payload is inserted into the payload receptacle 1304 until the bottom of the alignment tab 1312 rests on the bottom surface of the alignment feature 1314 of the payload receptacle 1304. After the universal payload 1300 is fully inserted into the payload receptacle 1304, a retaining screw 1320 is screwed through the through-hole 1316 of the host implant 1302. Upon complete threading of the retaining of the retaining screw 1320, the end of the screw rests on top of the alignment tab 1312 of the universal payload 1300 to thereby secure the universal payload 1300 within the host implant.

### Threaded

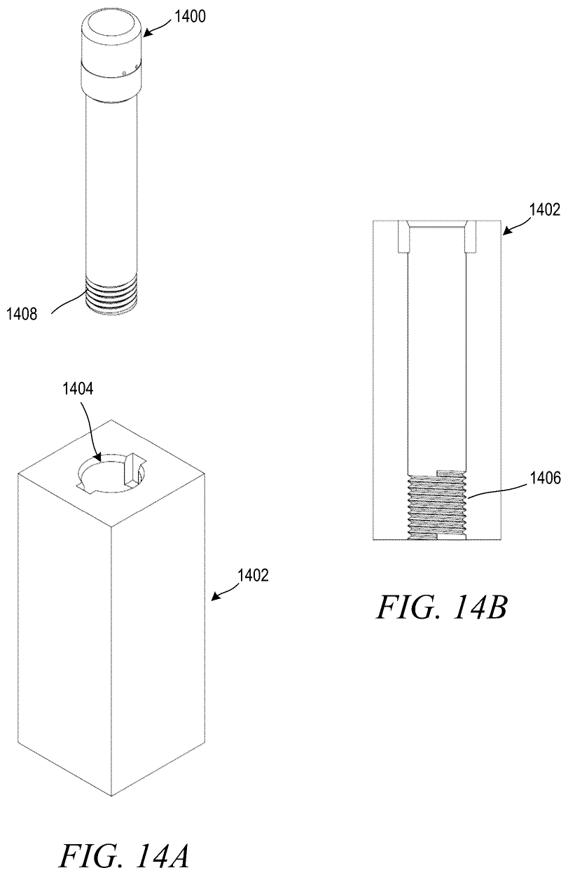

With reference to FIGS. 14A, 14B, and 14C, integration of a universal payload 1400 with a host implant 1402 can be enabled by respective securing features of the payload and implant, without the need for any separate components, e.g., retaining screws. While the host implant 1402 is generically illustrated as a block, the implant can be any one of the orthopedic implants described in the present disclosure.

In this embodiment, the host implant 1402 includes a payload receptacle 1404 or cavity bounded by an interior wall of the host implant. The payload receptacle 1404 has a form factor configured to receive a universal payload 1400. Internal threads 1406 are formed in the interior wall of the host implant 1402 at the bottom of the payload receptacle 1404. The lower region of the universal payload 1400 includes external threads 1408.

During integration of the universal payload 1400 with a host implant 1402, the universal payload 1400 is inserted into the payload receptacle 1404, and rotated to engage the external threads 1408 of the payload with the internal threads 1406 of the host implant 1402 to thereby secure the universal payload 1400 within the host implant.

### Reverse Threaded Connection

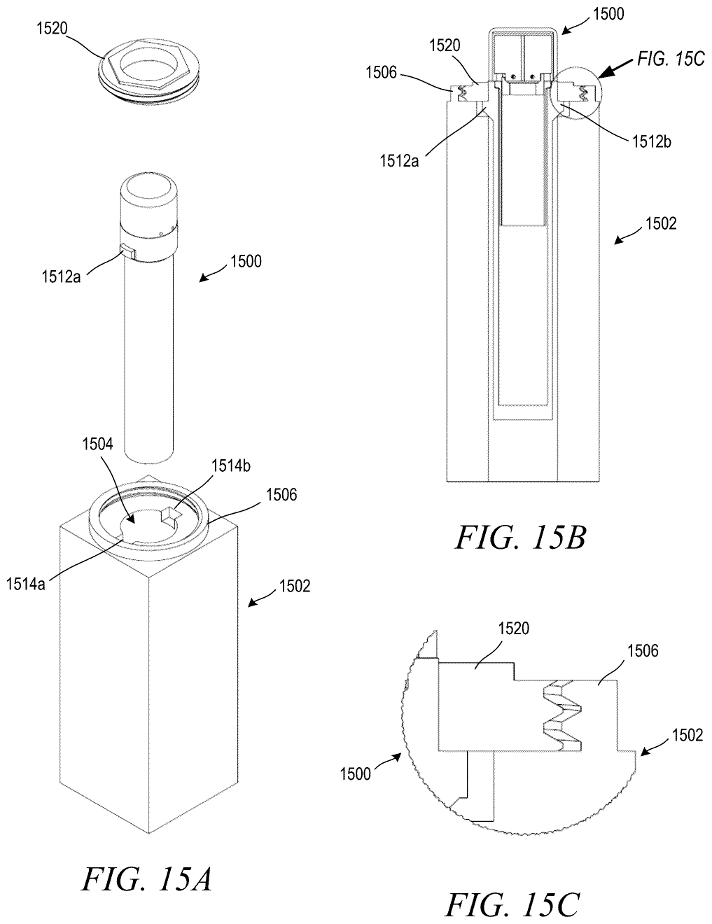

With reference to FIGS. 15A, 15B, and 15C, integration of a universal payload 1500 with a host implant 1502 can be enabled by respective alignment features of the payload and implant, respective securing features of the payload and implant, and an additional securing element, e.g., a hex-keyed fastener 1520 with external threads. While the host implant 1502 is generically illustrated as a block, the implant can be any one of the orthopedic implants described in the present disclosure.

In this embodiment, the host implant 1502 includes a payload receptacle 1504 or cavity bounded by an interior wall of the host implant. The payload receptacle 1504 has a form factor configured to receive a universal payload 1500. The host implant 1502 also includes a pair of alignment slots 1514a, 1514b and a ring 1506 with internal threads that surrounds the opening of the payload receptacle 1504. The ring 1506 is configured to receive the hex-keyed fastener 1520. The upper region of the universal payload 1500 includes a pair of alignment tabs 1512a, 1512b.

During integration of the universal payload 1500 with a host implant 1502, the alignment tabs 1512a, 1512b of the universal payload are aligned with the alignment slots 1514a, 1514b of the host implant, and the universal payload is inserted into the payload receptacle 1404 until the bottom of the alignment tabs 1512a, 1512b rest on the bottom surface of the alignment slots 1514a, 1514b of the payload receptacle 1504. After the universal payload 1500 is fully inserted into the payload receptacle 1504, a hex-keyed fastener 1520 is screwed into the ring 1506 to thereby secure the universal payload 1500 within the host implant 1502.

### Tapered Fit

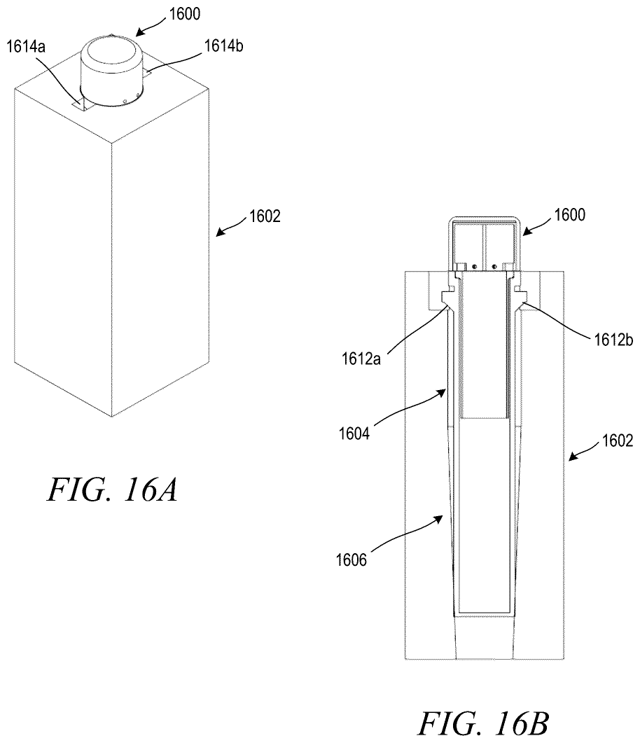

With reference to FIGS. 16A and 16B, integration of a universal payload 1600 with a host implant 1602 can be enabled by respective alignment features of the payload and implant, and respective securing features of the payload and implant, without the need for any separate components, e.g., retaining screws. While the host implant 1602 is generically illustrated as a block, the implant can be any one of the orthopedic implants described in the present disclosure.

In this embodiment, the host implant 1602 includes a payload receptacle 1604 or cavity bounded by an interior wall of the host implant. The payload receptacle 1604 has a form factor configured to receive a universal payload 1600 and includes a tapered region 1606 that narrows the form factor of the payload receptacle to size slightly less than the form factor of the lower region of the universal payload 1600. The host implant 1602 also includes a pair of alignment slots 1614a, 1614b. The upper region of the universal payload 1600 includes a pair of alignment tabs 1612a, 1612b.

During integration of the universal payload 1600 with a host implant 1602, the alignment tabs 1612a, 1612b of the universal payload are aligned with the alignment slots 1614a, 1614b of the host implant, and the universal payload is inserted into the payload receptacle 1604. As the universal payload 1600 is advanced into the payload receptacle 1604 the lower region of the payload engages the tapered region 1606 of the receptacle. The universal payload 1600 is then impacted further into the payload receptacle 1604 using an impaction tool until the bottom of the alignment tabs 1612a, 1612b rest on the bottom surface of the alignment slots 1614a, 1614b of the payload receptacle 1604. The friction fit between the tapered region 1606 of the payload receptacle 1604 and the lower region of the universal payload 1600 secures the universal payload 1500 within the host implant 1602.

### Nitinol Memory Wire

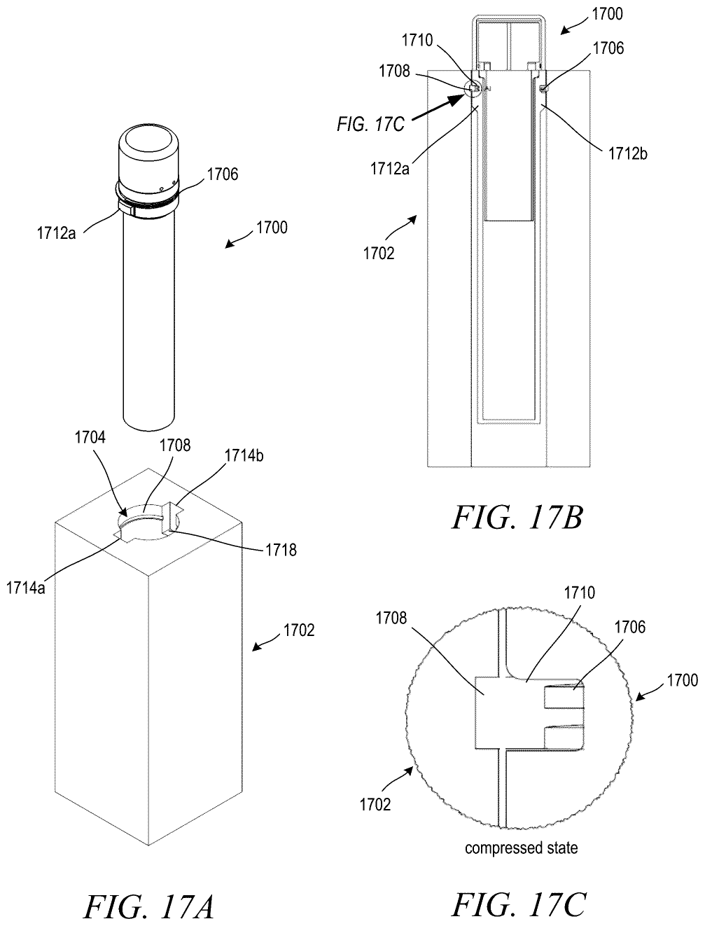

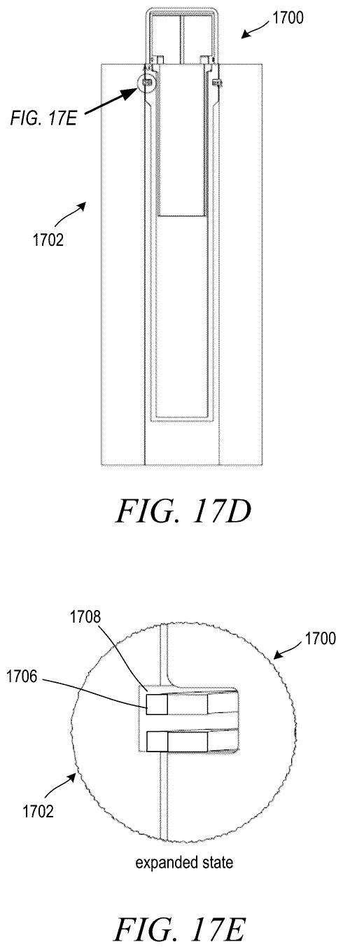

With reference to FIGS. 17A 17B, 17C, 17D, and 17E, integration of a universal payload 1700 with a host implant 1702 can be enabled by respective alignment features of the payload and implant, and respective securing features of the payload and implant, without the need for any separate components, e.g., retaining screws. While the host implant 1702 is generically illustrated as a block, the implant can be any one of the orthopedic implants described in the present disclosure.

In this embodiment, the host implant 1702 includes a payload receptacle 1704 or cavity bounded by an interior wall of the host implant. The payload receptacle 1704 has a form factor configured to receive a universal payload 1700. An annular groove 1708 is formed in the interior wall of the host implant 1702. The upper region of the universal payload 1700 includes a memory wire structure 1706 positioned in an annular groove 1710, and pair of alignment tabs 1712a, 1712b. The memory wire structure 1706 is configured as a coil and is formed of a material, e.g., Nitinol, that expands upon application of heat. Thus, the memory wire structure 1706 is configured to transition from a normally compressed state having a first diameter to an expanded state (upon application of heat) having a second diameter greater than the first diameter.

During integration of the universal payload 1700 with a host implant 1702, the alignment tabs 1712a, 1712b of the universal payload 1700 are aligned with corresponding alignment slots 1714a, 1714b of the host implant 1702, and the universal payload 1700 is inserted into the payload receptacle 1704 until the bottom of the alignment tabs 1712a, 1712b rest on a bottom surface 1718 the alignment slots 1714a, 1714b of the host implant 1702. After the universal payload 1700 is fully inserted into the payload receptacle 1704, the annular groove 1710 of the universal payload 1700 is aligned with the annular groove 1708 of the host implant 1702. Heat is then targeted to the memory wire structure 1706, which heat causes the wire structure to transition from its compressed state (FIGS. 17B and 17C) to its expanded state (FIGS. 17D and 17E) to thereby secure the universal payload within the host implant.

### Over-Mold Snap-Fit on Cartridge

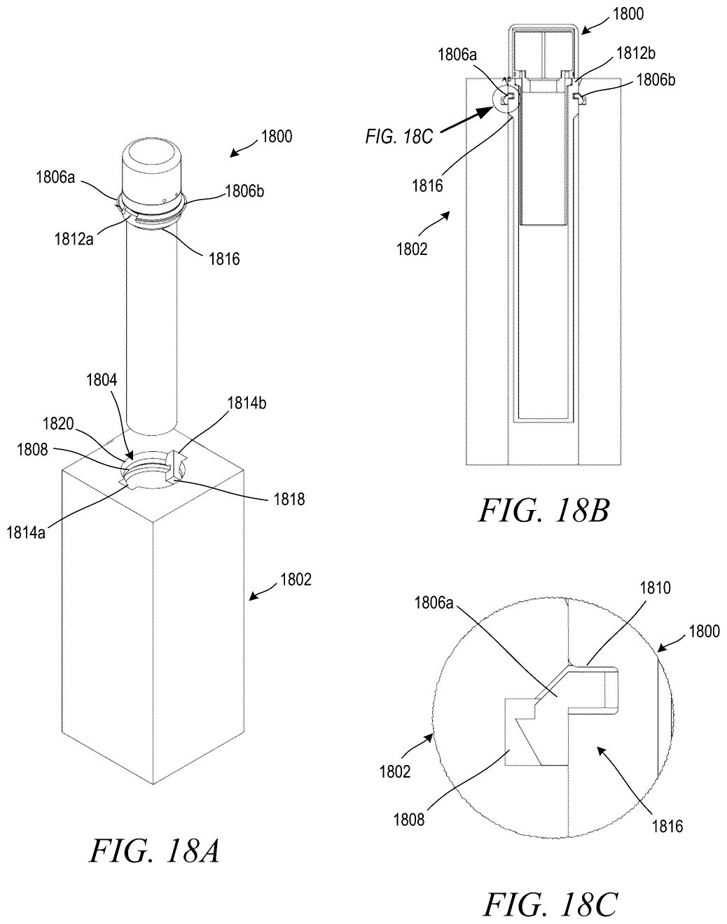

With reference to FIGS. 18A, 18B, and 18C, integration of a universal payload 1800 with a host implant 1802 can be enabled by respective alignment features of the payload and implant, and respective securing features of the payload and implant, without the need for any separate components, e.g., retaining screws. While the host implant 1802 is generically illustrated as a block, the implant can be any one of the orthopedic implants described in the present disclosure.

In this embodiment, the host implant 1802 includes a payload receptacle 1804 or cavity bounded by an interior wall of the host implant. The payload receptacle 1804 has a form factor configured to receive a universal payload 1800. An annular groove 1808 is formed in the interior wall of the host implant 1802. The upper region of the universal payload 1800 includes a pair of over-mold snap structures 1806a, 1806b positioned partially in an annular groove 1810 and partially adjacent a shoulder 1816 of the universal payload. The portions of the shoulder 1816 between the pair of over-mold snap structures 1806a, 1806b define respective alignment tabs 1812a, 1812b. Each of the over-mold snap structures 1806a, 1806b is configured as an arcuate structure formed of a material, e.g., plastic, that flexes upon application of force.

During integration of the universal payload 1800 with a host implant 1802, the alignment tabs 1812a, 1812b of the universal payload 1800 are aligned with corresponding alignment slots 1814a, 1814b of the host implant 1802, and the universal payload 1800 is inserted into the payload receptacle 1804 until the bottom of the alignment tabs 1812a, 1812b rest on a bottom surface 1818 of the alignment slots 1814a, 1814b of the host implant 1802. During insertion of the universal payload 1800 the over-mold snap structures 1806a, 1806b engage a tapered rim 1820 of the payload receptacle 1804. Upon application of insertion force on the universal payload 1800, the tapered rim 1820 causes the over-mold snap structures 1806a, 1806b to flex and snap fit into the annular groove 1808 of the host implant 1802 to thereby secure the universal payload within the host implant.

Collet with Tapered Interface

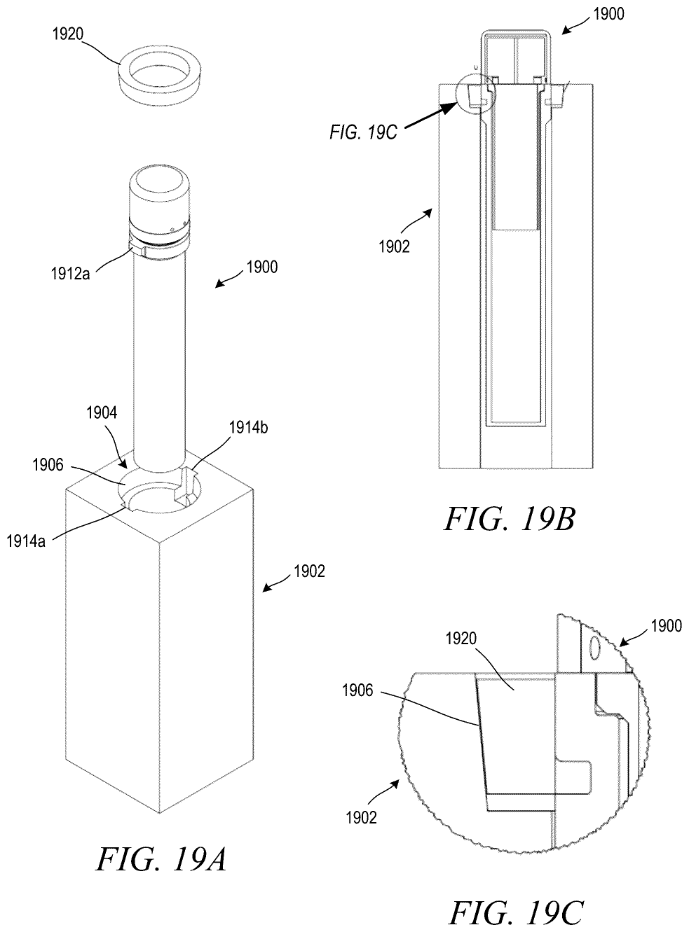

With reference to FIGS. 19A, 19B, and 19C, integration of a universal payload 1900 with a host implant 1902 can be enabled by respective alignment features of the payload and implant, respective securing features of the payload and implant, and an additional securing element, e.g., a collet 1920 with a tapered exterior wall. While the host implant 1902 is generically illustrated as a block, the implant can be any one of the orthopedic implants described in the present disclosure.

In this embodiment, the host implant 1902 includes a payload receptacle 1904 or cavity bounded by an interior wall of the host implant. The payload receptacle 1904 has a form factor configured to receive a universal payload 1900. The host implant 1902 also includes a pair of alignment slots 1914a, 1914b and a tapered interior sidewall 1906 at the top portion of the payload receptacle 1904. The upper region of the universal payload 1900 includes a pair of alignment tabs 1912a, 1912b.

During integration of the universal payload 1900 with a host implant 1902, the alignment tabs 1912a, 1912b of the universal payload are aligned with the alignment slots 1914a, 1914b of the host implant, and the universal payload is inserted into the payload receptacle 1404 until the bottom of the alignment tabs 1912a, 1912b rest on the bottom surface of the alignment slots 1914a, 1914b of the payload receptacle 1904. After the universal payload 1900 is fully inserted into the payload receptacle 1904, the collet 1920 is impacted into the top portion of the of the payload receptacle 1904 having the tapered interior sidewall 1906, to thereby establish a tight, friction fit between the tapered interior sidewall 1906 and the tapered exterior wall of the collet 1920 that secures the universal payload 1900 within the host implant 1902.

### Form Factors

As previously noted, “form factor” as used herein refers to physical characteristics, e.g., three-dimensional shape and size, of a structure or empty space. The universal payload 100 of FIGS. 1A-19C is characterized by a generally cylindrical form factor having circular cross-sections along its length. A cylindrical form factor with circular cross-sections (as opposed to a block form factor with square or rectangular cross-sections) provides the smallest form factor for a given volume. While the cylindrical form factor with circular cross-section provides the smallest form factor, which may be a necessary consideration in selecting the form factor of the universal payload, form factors having non-circular cross-sections may alternatively be employed. In some instances, a form factor having non-circular cross-sections, referred to going forward as “a non-circular form factor” may reduce the number of orientations in which the universal payload may fit into the receptacle of the implant, and thereby assist in creating a known and fixed orientation of the universal payload in the implant. For example, a universal payload with an oval form factor will have only a single orientation which will fit within the receptacle of the implant, where that single orientation effectively locks the universal payload in place, i.e., the payload can only fit in the implant in only a single orientation, and cannot rotate and thereby change orientation after being positioned in the receptacle, thus providing for a known and constant alignment of the payload in the implant.

Other form factors may also be employed for similar purposes, e.g., triangle, square, rectangle, kite, trapezoid, parallelogram and rhombus are suitable form factors for the universal payload and corresponding payload receptacle of a host implant. Depending on the design of the form factor, the universal payload may fit into the payload receptacle in only one, or two, or three, or four, or five, or six, etc. orientations, thus reducing the number of possible orientations compared to those provided by a circular cross-section form factor. In one embodiment the form factor has a plane of symmetry, while in another embodiment the form factor does not have a plane of symmetry.

The form factor may or may not have the same cross-section along its entire length where the housing of the universal payload abuts the interior surface of the payload receptacle of the host implant. For example, the form factor may have a circular cross section toward the proximal end of the payload/receptacle abutment, but may change to having a non-circular cross section toward the distal end of the payload/receptacle abutment. The non-circular distal end of the payload/receptacle abutment may provide for only a single orientation whereby the universal payload can be seated in the payload receptacle. Alternatively, the non-circular distal end of the payload/receptacle abutment may provide for only two, or three, or four, or five, or six, etc. possible orientations when the universal payload is seated within the implant receptacle.

The non-circular distal end of the payload/receptacle abutment may provide for stability, or enhanced stability, of the payload/receptacle alignment, in the event that the payload may not rotate within the receptacle due the rotation causing a surface of the payload to abut against an immovable surface of the implant. As one example, the distal end of the payload may have a cross section in the shape of a plus sign, and the distal end of the receptacle may have a cross section that is the complement of a plus sign, so that the distal end of the payload fits securely into the distal end of the receptacle and the entirely of the payload cannot rotate within the receptacle. The four arms of the plus sign need not be exactly the same size or exactly the same shape, so that there is but a single orientation in which the payload fully seats in the receptacle.

### Universal Payload Electronics

With reference to FIG. 20A, a universal payload 2000 includes an electronics assembly 2010, a battery 2012 or other suitable implantable power source, an antenna 2030, and one or more external sensors 2040. The electronics assembly 2010 includes a fuse 2014, switches 2016 and 2018, a clock generator and clock and power management circuit 2020, an inertial measurement unit (IMU) 2022 having accelerometers and gyroscopes, a memory circuit 2024, a radio-frequency (RF) transceiver 2026, an RF filter 2028 and a controller 2032 with at least two joint-specific modules, e.g., a knee module 2050, a hip module 2052, and a shoulder module 2054. Although not shown in FIG. 20A, the electronics assembly 2010 may also include an accelerometer that functions independent of the accelerometers of the IMU. Examples of some of these components are described elsewhere in this application and in PCT Publication No. WO 2022/266085.

The battery 2012 can be any suitable battery, such as a Lithium Carbon Monofluoride (LiCFx) battery, or other storage cell configured to store energy for powering the electronics assembly 2010 for an expected lifetime (e.g., 5-25+ years) of the kinematic implant.

The fuse 2014 can be any suitable fuse (e.g., permanent) or circuit breaker (e.g., resettable) configured to prevent the battery 2012, or a current flowing from the battery, from injuring the patient and damaging the battery and one or more components of the electronics assembly 2010. For example, the fuse 2014 can be configured to prevent the battery 2012 from generating enough heat to burn the patient, to damage the electronics assembly 2010, to damage the battery, or to damage structural components of the kinematic implant.

The switch 2016 is configured to couple the battery 2012 to, or to uncouple the battery from, the IMU 2022 in response to a control signal from the controller 2032. For example, the controller 2032 may be configured to generate the control signal having an open state that causes the switch 2016 to open, and, therefore, to uncouple power from the IMU 2022, during a sleep mode or other low-power mode to save power, and, therefore, to extend the life of the battery 2012. Likewise, the controller 2032 also may be configured to generate the control signal having a closed state that causes the switch 2016 to close, and therefore, to couple power to the IMU 2022, upon “awakening” from a sleep mode or otherwise exiting another low-power mode. Such a low-power mode may be for only the IMU 2022 or for the IMU and one or more other components of the implantable.

The switch 2018 is configured to couple the battery 2012 to, or to uncouple the battery from, the memory circuit 2024 in response to a control signal from the controller 2032. For example, the controller 2032 may be configured to generate the control signal having an open state that causes the switch 2018 to open, and, therefore, to uncouple power from the memory circuit 2024, during a sleep mode or other low-power mode to save power, and, therefore, to extend the life of the battery 2012. Likewise, the controller 2032 also may be configured to generate the control signal having a closed state that causes the switch 2018 to close, and therefore, to couple power to the memory circuit 2024, upon “awakening” from a sleep mode or otherwise exiting another low-power mode. Such a low-power mode may be for only the memory circuit 2024 or for the memory circuit and one or more other components of the electronics assembly 2010.

The clock and power management circuit 2020 can be configured to generate a clock signal for one or more of the other components of the electronics assembly 2010, and can be configured to generate periodic commands or other signals (e.g., interrupt requests) in response to which the controller 2032 causes one or more components of the implantable circuit to enter or to exit a sleep, or other low-power, mode. The clock and power management circuit 2020 also can be configured to regulate the voltage from the battery 2012, and to provide a regulate power-supply voltage to some or all of the other components of the electronics assembly 2010.

The IMU 2022 has a frame of reference with coordinate x, y, and z axes, and can be configured to measure, or to otherwise quantify, acceleration that the IMU experiences along each of the x, y, and z axes, and angular velocity that the IMU experiences about each of the x, y, and z axes. Such a configuration of the IMU 2022 is at least a six-axis configuration, because the IMU 2022 measures six unique quantities, accₓ(t), acc_{y}(t), acc_{z}(t), Ωₓ(t), Ω_{y}(t), and Ω_{z}(t). Alternatively, the IMU 2022 can be configured in a nine-axis configuration, in which the IMU can use gravity to compensate for, or to otherwise correct for, accumulated errors in accₓ(t), acc_{y}(t), acc_{z}(t), Ωₓ(t), Ω_{y}(t), and Ω_{z}(t). But in an embodiment in which the IMU measures acceleration and angular velocity over only short bursts (e.g., 0.10-100 seconds(s)), for many applications accumulated error typically can be ignored without exceeding respective error tolerances.

The IMU 2022 can include a respective analog-to-digital converter (ADC) for each of the x, y, and z accelerometers and gyroscopes. Alternatively, the IMU 2022 can include a respective sample-and-hold circuit for each of the x, y, and z accelerometers and gyroscopes, and as few as one ADC that is shared by the accelerometers and gyroscopes. Including fewer than one ADC per accelerometer and gyroscope can decrease one or both of the size and circuit density of the IMU 2022, and can reduce the power consumption of the IMU. But because the IMU 2022 includes a respective sample-and-hold circuit for each accelerometer and each gyroscope, samples of the analog signals generated by the accelerometers and the gyroscopes can be taken at the same or different sample times, at the same or different sample rates, and with the same or different output data rates (ODR).

As mentioned above, the electronics assembly 2010 may also include an accelerometer that functions independent of the accelerometers of the IMU. This accelerometer may be configured to monitor acceleration in a low power state. The accelerometer may be a single axis or multi-axis accelerometer, and in one embodiment is a triaxial accelerometer. Based on acceleration signals it senses, this accelerometer can detect motion events. For example, the accelerometer can be configured to detect simple motion events, such as footsteps or shoulder swings, and to count such detections. The accelerometer can be configured to detect significant motion, such as a walking motion or arm swinging motion. The accelerometer may be configured to provide a wake-up signal to the controller 1032 when significant motion is detected.

The memory circuit 2024 can be any suitable nonvolatile memory circuit, such as EEPROM or FLASH memory, and can be configured to store data written by the controller 2032, and to provide data in response to a read command from the controller.

The RF transceiver 2026 can be a conventional transceiver that is configured to allow the controller 2032 (and optionally the fuse 2014) to communicate with a base station configured for use with the kinematic implantable device. For example, the RF transceiver 2026 can be any suitable type of transceiver (e.g., Bluetooth, Bluetooth Low Energy (BTLE), and WiFi®), can be configured for operation according to any suitable protocol (e.g., MICS, ISM, Bluetooth, Bluetooth Low Energy (BTLE), and WiFi®), and can be configured for operation in a frequency band that is within a range of 1 MHz-5.4 GHz, or that is within any other suitable range.

The RF filter 2028 can be any suitable bandpass filter, such as a surface acoustic wave (SAW) filter or a bulk acoustic wave (BAW) filter. In some embodiment, the RF filter 1028 includes multiple filters and other circuitry to enable dual-band communication. For example, the RF filter 1028 may include a bandpass filter for communications on a MICS channel, and a notch filter for communication on a different channel, such as a 2.45 GHz.

The antenna 2030 can be any antenna suitable for the frequency band in which the RF transceiver 2026 generates signals for transmission by the antenna, and for the frequency band in which a base station generates signals for reception by the antenna.

The external sensors 2040 may include, for example, chemistry sensors (e.g., for blood and/or other fluids), metabolic sensors (e.g., for blood and/or other fluids), analyte sensors (e.g., for glucose), pH sensors, temperature sensors, fluid pressure sensors, fluid volume sensors, contact sensors, and position sensors. The external sensors 2040 may be located on the exterior of the cap 116 of the antenna assembly 112 to provide contact with biological fluid or tissue, or exposure to an intrabody space around the antenna assembly. The external sensors 2040 may couple to the electronics assembly 2010 through a feedthrough through the cap 116.

The controller 2032, which can be any suitable microcontroller or microprocessor, is configured to control the configuration and operation of one or more of the other components of the electronics assembly 2010. For example, different modules of the controller 2032 are configured to control the IMU 2022 to take measurements of movement of the host implant with which the universal payload 2000 is integrated, to quantify the quality of such measurements (e.g., is the measurement “good” or “bad”), to store measurement data generated by the IMU in the memory circuit 2024, to generate messages that include the stored data, to packetize the messages, to provide the message packets to the RF transceiver 2026 for transmission to an external device, e.g. a base station.

The controller 2032 may be configured to execute commands received from an external device via the antenna 2030, the RF filter 2028, and the RF transceiver 2026. For example, the controller 2032 can be configured to receive configuration data from a base station, and to provide the configuration data to the component of the electronics assembly 2010 to which the base station directed the configuration data. If the base station directed the configuration data to the controller 2032, then the controller is configured to configure itself in response to the configuration data. The controller 2032 may also be configured to execute data sampling by the IMU 2022 in accordance with one or more programmed sampling schedules, or in response to an on-demand data sampling command received from a base station.

In some embodiments, the controller 2032 includes a knee module 2050, a hip module 2052, and a shoulder module 2054. Each of these modules may comprise firmware and/or software configured to operate the universal payload 2000 is a manner specific to the host orthopedic implant in which the universal payload is integrated. The knee module 2050 is configured to operate the IMU 2022 to obtain kinematic information associated with motion of a knee implant. The knee module 2050 include two submodules, one that operate the IMU 2022 to obtain kinematic information associated with motion of a left knee implant, and a second that operate the IMU 2022 to obtain kinematic information associated with motion of a right knee implant.

The hip module 2052 is configured to operate the IMU 2022 to obtain kinematic information associated with motion of a hip implant. The hip module 2052 include two submodules, one that operate the IMU 2022 to obtain kinematic information associated with motion of a left hip implant, and a second that operate the IMU 2022 to obtain kinematic information associated with motion of a right hip implant. The shoulder module 2054 is configured to operate the IMU 2022 to obtain kinematic information associated with motion of a shoulder implant. The shoulder module 2054 include two submodules, one that operate the IMU 2022 to obtain kinematic information associated with motion of a left shoulder implant, and a second that operate the IMU 2022 to obtain kinematic information associated with motion of a right shoulder implant.

Depending on the type of host orthopedic implant in which the universal payload 2000 is integrated, one submodule of these joint-specific modules 2050, 2052, 2054 is activated, while the other submodules are disabled.

### Operation of Universal Payload

Continuing with FIG. 20A, operation of a universal payload 2000 is now described. The fuse 2014, which is normally electrical closed, is configured to open electrically in response to an event that can injure the patient in which the intelligent implant 2000 resides, or damage the battery 2012 of the intelligent implant if the event persists for more than a safe length of time. An event in response to which the fuse 2014 can open electrically includes an overcurrent condition, an overvoltage condition, an overtemperature condition, an over-current-time condition, and over-voltage-time condition, and an over-temperature-time condition. An overcurrent condition occurs in response to a current through the fuse 2014 exceeding an overcurrent threshold. Likewise, an overvoltage condition occurs in response to a voltage across the fuse 2014 exceeding an overvoltage threshold, and an overtemperature condition occurs in response to a temperature of the fuse exceeding a temperature threshold. An over-current-time condition occurs in response to an integration of a current through the fuse 2014 over a measurement time window (e.g., ten seconds) exceeding a current-time threshold, where the window can “slide” forward in time such that the window always extends from the present time back the length, in units of time, of the window. Alternatively, an over-current-time condition occurs if the current through the fuse 2014 exceeds an overcurrent threshold for more than a threshold time.

Similarly, an over-voltage-time condition occurs in response to an integration of a voltage across the fuse 2014 over a measurement time window, and an over-temperature-time condition occurs in response to an integration of a temperature of the fuse over a measurement time window. Alternatively, an over-voltage-time condition occurs if the voltage across the fuse 2014 exceeds an overvoltage threshold for more than a threshold time, and an over-temperature-time condition occurs if a temperature associated with the fuse 2014, battery 2012, or electronics assembly 2010 exceeds an overtemperature threshold for more than a threshold time. But even if the fuse 2014 opens, thus uncoupling power from the electronics assembly 2010, the mechanical and structural components of the universal payload 2000 is associated are still fully operational. For example, if the universal payload 2000 is a knee prosthesis, then the knee prosthesis still can function fully as a patient's knee; abilities lost, however, are the abilities to detect and to measure kinematic motion of the prosthesis, to generate and to store data representative of the measured kinematic motion, and to provide the stored data to a base station or other destination external to the kinematic prosthesis.

In some embodiments, a joint-specific module 2050, 2052, 2054 is configured to cause the IMU 2022 to measure, in response to a movement of the universal payload 2000, movement of a joint over a window of time (e.g., ten seconds, twenty seconds, one minute), to determine if the measured movement is a qualified movement, to store the data representative of a measured qualified movement, and to cause the RF transceiver 2026 to transmit the stored data to a base station or other source external to the prosthesis.

For example, in the case of a host knee implant, the knee module 2050 may control the operation of the IMU 1022 such that the IMU begins sampling the sense signals output from its one or more accelerometers and one or more gyroscopes in response to a detected movement within a respective time period (day), and the knee module 2050 can analyze the samples to determine if the detected movement is a qualified movement. Further in example, the IMU 2022 can detect movement in any conventional manner, such as by movement of one or more of its one or more accelerometers. In response to the IMU 2022 notifying knee module 2050 of the detected movement, the knee module can correlate the samples from the IMU to stored accelerator and gyroscope samples generated with a computer simulation or while the patient, or another patient, is walking normally, and can measure the time over which the movement persists (the time equals the number of samples times the inverse of the sampling rate). If the samples of the accelerator and gyroscope output signals correlate with the respective stored samples, and the time over which the movement persists is greater than a threshold time, then the knee module 2050 effectively labels the movement as a qualified movement.

In response to determining that the movement is a qualified movement, the knee module 2050 stores the samples, along with other data, in the memory circuit 2024, and may disable the IMU 2022 until the next time period (e.g., the next day or the next week) by opening the switch 2016 to extend the life of the battery 2012. The clock and power management circuit 2020 can be configured to generate periodic timing signals, such as interrupts, to commence each time period. For example, the knee module 2050 can close the switch 2016 in response to such a timing signal from the clock and power management circuit 2020. Furthermore, the other data can include, e.g., the respective sample rate for each set of accelerometer and gyroscope samples, a respective time stamps indicating the time at which the IMU 2022 acquired the corresponding sets of samples, the respective sample times for each set of samples, an identifier (e.g., serial number) of the implantable prosthesis, and a patient identifier (e.g., a number or name). The volume of the other data can be significantly reduced if the sample rate, time stamp, and sample time are the same for each set of samples (i.e., samples of signals from all accelerometers and gyroscopes taken at the same times at the same rate) because the header includes only one sample rate, one time stamp, and one set of sample times for all sets of samples. Furthermore, the knee module 2050 can encrypt some or all of the data in a conventional manner before storing the data in the memory circuit 2024. For example, the knee module 2050 can encrypt some or all of the data dynamically such that at any given time, the same data has a different encrypted form than if encrypted at another time.

The stored data samples of the signals that the one or more accelerometers and one or more gyroscopes of the IMU 2022 generate can provide clues to the condition of the implantable prosthesis. For example, one can analyze the data samples (e.g., with a remote server such as a cloud server) to determine whether a surgeon implanted the prosthesis correctly, to determine the level(s) of instability and degradation that the implanted prosthesis exhibits at present, to determine the instability and degradation profiles over time, and to compare the instability and degradation profiles to benchmark instability and degradation profiles developed with stochastic simulation or data from a statistically significant group of patients.

Furthermore, the sampling rate, output data rate (ODR), and sampling frequency of the IMU 2022 can be configured to any suitable values. For example, the knee module 2050 may control the operation of IMU 2022 so that the sampling rate may be fixed to any suitable value such as at 3200 Hz, the ODR, which can be no greater than the sampling rate and which is generated by “dropping” samples periodically, can be any suitable value such as 800 Hz, and the sampling frequency (the inverse of the interval between sampling periods) for qualified events can be any suitable value, such as twice per day, once per day, once per every 2 days, once per week, once per month, or more or less frequently. And sampling rate or ODR can be varied depending on the type of event being sampled. For example, to detect that the patient is walking without analyzing the patient's gait or the implant for instability or wear, the sampling rate or ODR can be 200 Hz, 25 Hz, or less. Therefore, such a low-resolution mode can be used to detect a precursor (a patient taking steps with a knee prosthesis) to a qualified event (a patient taking at least ten consecutive steps) because a “search” for a qualified event may include multiple false detections before the qualified even is detected. By using a lower sampling rate or ODR, the IMU 1022 saves power while conducting the search, and increases the sampling rate or the ODR (e.g., to 800 Hz, 1600, or 3200 Hz) only for sampling a detected qualified event so that the accelerator and gyroscope signals have sufficient sampling resolution for analysis of the samples for, e.g., instability and wear of the prosthesis.

While the foregoing speaks in terms of the knee module 2050 of the universal payload, similar operations may be performed by the hip module 2052 or the shoulder module 2054.

Systems with Intelligent Implants

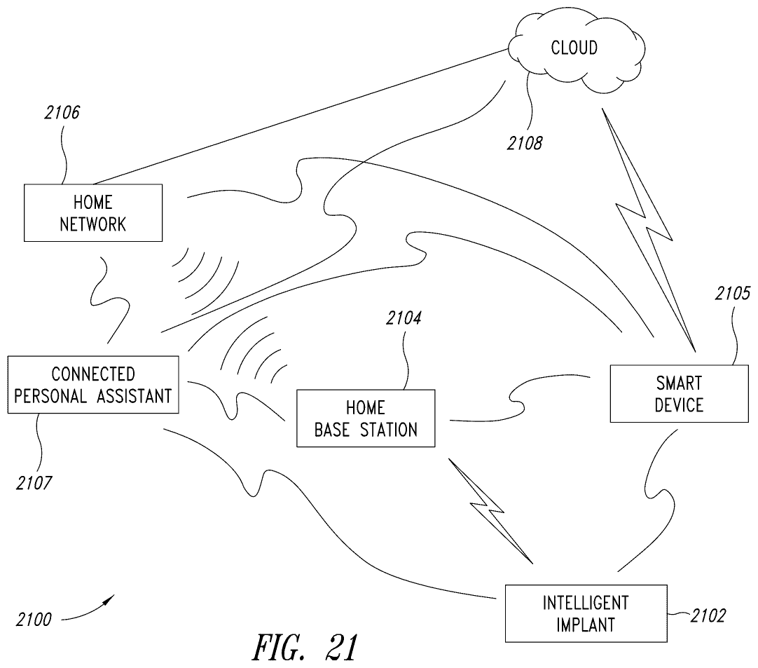

FIG. 21 illustrates a context diagram of an operation environment 2100. In the environment, a host orthopedic implant having an integrated universal payload 100 (referred to from hereon as an intelligent implant 2102) is implanted by a medical practitioner in the body of a patient The intelligent implant 2102 is arranged to collect data including operational data of the device along with kinematic data associated with particular movement of the patient or particular movement of a portion of the patient's body, for example, one of the left knee or the right knee of the patient. The intelligent implant 2102 communicates with one or more base stations or one or more smart devices during different stages of monitoring the patient.

For example, in association with a medical procedure, an intelligent implant 2102 is implanted in the patient's body. Coetaneous with the medical procedure, the intelligent implant 2102 communicates with an operating room base station during which the appropriate one of the knee module 2050, the hip module 2052, and the shoulder module 2054 is activated. Subsequently, after sufficient recovery from the medical procedure, the patient returns home wherein the intelligent implant 2102 is arranged to communicate with a home base station 2104. At other times, the intelligent implant 2102 is arranged to communicate with a doctor office base station. The intelligent implant 2102 communicates with each base station via a short-range network protocol, such as the medical implant communication service (MICS), the medical device radio communications service (MedRadio), or some other wireless communication protocol suitable for use with the intelligent implant 2102.

The intelligent implant 2102 includes one or more sensors to collect information and kinematic data associated with the use of the body part to which the intelligent implant 2102 is associated. For example, the intelligent implant 2102 may include an inertial measurement unit that includes gyroscope(s), accelerometer(s), pedometer(s), or other kinematic sensors to collect acceleration data for the medial/lateral, anterior/posterior, and anterior/inferior axes of the associated body part; angular velocity for the sagittal, frontal, and transvers planes of the associated body part; force, stress, tension, pressure, duress, migration, vibration, flexure, rigidity, or some other measurable data.

The intelligent implant 2102 collects data at various different times and at various different rates during a monitoring process of the patient. In some embodiments, the intelligent implant 2102 may operate in a plurality of different phases over the course of monitoring the patient so that more data is collected soon after the intelligent implant 2102 is implanted into the patient, but less data is collected as the patient heals and thereafter.

In one non-limiting example, the monitoring process of the intelligent implant 2102 may include three different phases. A first phase may last for four months where kinematic data is collected once a day for one minute, every day of the week. After the first phase, the intelligent implant 2102 transitions to a second phase that lasts for eight months and collects kinematic data once a day for one minute, two days a week. And after the second phase, the intelligent implant 2102 transitions to a third phase that lasts for nine years and collects kinematic data one day a week for one minute for the next nine years. Of course, the time periods associated with each phase may be longer, shorter, and otherwise controllable; for example, the time periods can be selected to be compatible with time periods specified by medical-insurance telemedicine codes so that a physician billing under telemedicine codes can collect the maximum reimbursement allowed by a medical insurer. The type and amount of data collected may also be controllable. The added benefit of this passive monitoring process is that after the first phase of monitoring, the patient will be unaware of when data is being collected. Thus, the collected data will be protected from potential bias.

Along with the various different phases, the intelligent implant 2102 can operate in various modes to detect different types of movements. In this way, when a predetermined type of movement is detected, the intelligent implant 2102 can increase, decrease, or otherwise control the amount and type of kinematic data and other data that is collected.

In one example, the intelligent implant 2102 may use a pedometer to determine if the patient is walking. If the intelligent implant 2102 measures that a determined number of steps crosses a threshold value within a predetermined time, then the intelligent implant 2102 may determine that the patient is walking. In response to the determination, the amount and type of data collected can be started, stopped, increased, decreased, or otherwise suitably controlled. The intelligent implant 2102 may further control the data collection based on certain conditions, such as when the patient stops walking, when a selected maximum amount of data is collected for that collection session, when the intelligent implant 2102 times out, or based on other conditions. After data is collected in a particular session, the intelligent implant 2102 may stop collecting data until the next day, the next time the patient is walking, after previously collected data is offloaded (e.g., by transmitting the collected data to the home base station 2104), or in accordance with one or more other conditions.

The amount and type of data collected by an intelligent implant 2102 may be different from patient to patient, and the amount and type of data collected may change for a single patient. For example, a medical practitioner studying data collected by the intelligent implant 2102 of a particular patient may adjust or otherwise control how the intelligent implant 2102 collects future data.

The amount and type of data collected by an intelligent implant 2102 may be different for different body parts, for different types of movement, for different patient demographics, or for other differences. Alternatively, or in addition, the amount and type of data collected may change overtime based on other factors, such as how the patient is healing or feeling, how long the monitoring process is projected to last, how much battery power remains and should be conserved, the type of movement being monitored, the body part being monitored, and the like. In some cases, the collected data is supplemented with personally descriptive information provided by the patient such as subjective pain data, quality of life metric data, co-morbidities, perceptions, or expectations that the patient associates with the intelligent implant 2102, or the like.

In some embodiments, the intelligent implant 2102 is implanted into a patient to monitor movement or other aspects of a particular body part. Implantation of the intelligent implant 2102 into the patient may occur in an operating room. As used herein, an operating room includes any office, room, building, or facility where the intelligent implant 2102 is implanted into the patient. For example, the operating room may be a typical operating room in a hospital, an operating room in a surgical clinic or a doctor's office, or any other operating theater where the intelligent implant 2102 is implanted into the patient.

Once the intelligent implant 2102 is implanted into the patient and the patient returns home, the home base station 2104, the smart device 2105 (e.g., the patient's smart phone), the connected personal assistant 2107, or two or more of the home base station, and the smart device, and the connected personal assistant can communicate with the intelligent implant 2102. The intelligent implant 2102 can collect kinematic data at determined rates and times, variable rates and times, or otherwise controllable rates and times. Data collection can start when the intelligent implant 2102 is initialized in the operating room, when directed by a medical practitioner, or at some later point in time. At least some data collected by the intelligent implant 2102 may be transmitted to the home base station 2104 directly, to the smart device 2105 directly, to the connected personal assistant 2107 directly, to the base station via one or both of the smart device and the connected personal assistant, to the smart device via one or both of the base station and the connected personal assistant, or to the connected personal assistant via one or both of the smart device and the base station. Here, “one or both” means via an item alone, and via both items serially or in parallel. For example, data collected by the intelligent implant 2102 may be transmitted to the home base station 2104 via the smart device 2105 alone, via the connected personal assistant 2107 alone, serially via the smart device and the connected personal assistant, serially via the connected personal assistant and the smart device, and directly, and possibly contemporaneously, via both the smart device and the connected personal assistant.

Similarly, data collected by the intelligent implant 2102 may be transmitted to the smart device 2105 via the home base station 2104 alone, via the connected personal assistant 2107 alone, serially via the home base station and the connected personal assistant, serially via the connected personal assistant and the home base station, and directly, and possibly contemporaneously, via both the home base station and the connected personal assistant. Further in example, data collected by the intelligent implant 2102 may be transmitted to the connected personal assistant 2107 via the smart device 2105 alone, via the home base station 2104 alone, serially via the smart device and the home base station, serially via the home base station and the smart device, and directly, and possibly contemporaneously, via both the smart device and the home base station.

In various embodiments, one or more of the home base station 2104, the smart device 2105, and the connected personal assistant 2107 pings the intelligent implant 2102 at periodic, predetermined, or other times to determine if the intelligent implant 2102 is within communication range of one or more of the home base station, the smart device, and the connected personal assistant. Based on a response from the intelligent implant 2102, one or more of the home base station 2104, the smart device 2105, and the connected personal assistant 2107 determines that the intelligent implant 2102 is within communication range, and the intelligent implant 2102 can be requested, commanded, or otherwise directed to transmit the data it has collected to one or more of the home base station 2104, the smart device 2105, and the connected personal assistant 2107.

Along with transmitting collected data to the cloud 2108, one or more of the home base station 2104, the smart device 2105, and the connected personal assistant 2107 may also obtain data, commands, or other information from the cloud 2108 directly or via the home network 2106. One or more of the home base station 2104, the smart device 2105, and the connected personal assistant 2107 may provide some or all of the received data, commands, or other information to the intelligent implant 2102. Examples of such information include, but are not limited to, updated configuration information, diagnostic requests to determine if the intelligent implant 2102 is functioning properly, data collection requests, and other information.

The cloud 2108 may include one or more server computers or databases to aggregate data collected from the intelligent implant 2102, and in some cases personally descriptive information collected from a patient, with data collected from other intelligent implants (not illustrated), and in some cases personally descriptive information collected from other patients. In this way, the cloud 2108 can create a variety of different metrics regarding collected data from each of a plurality of intelligent implants that are implanted into separate patients. This information can be helpful in determining if the intelligent implants are functioning properly. The collected information may also be helpful for other purposes, such as determining which specific devices may not be functioning properly, determining if a procedure or condition associated with the intelligent implant is helping the patient (e.g., if the knee replacement is operating properly and reducing the patient's pain), and determining other medical information.

At various times throughout the monitoring process, the patient may be requested to visit a medical practitioner for follow up appointments. This medical practitioner may be the surgeon who implanted the intelligent implant 2102 in the patient or a different medical practitioner that supervises the monitoring process, physical therapy, and recovery of the patient. For a variety of different reasons, the medical practitioner may want to collect real-time data from the intelligent implant 2102 in a controlled environment. In some cases, the request to visit the medical practitioner may be delivered through a respective optional bidirectional user interface of each of one or more of the home base station 2104, the smart device 2105, and the connected personal assistant 2107.

A medical practitioner utilizes the doctor office base station, which communicates with the intelligent implant 2102, to pass additional data between the doctor office base station and the intelligent implant 2102. Alternatively, or in addition, the medical practitioner utilizes the doctor office base station to pass commands to the intelligent implant 2102. In some embodiments, the doctor office base station instructs the intelligent implant 2102 to enter a high-resolution mode to temporarily increase the rate or type of data that is collected for a short time. The high-resolution mode directs the intelligent implant 2102 to collect different (e.g., large) amounts of data during an activity where the medical practitioner is also monitoring the patient.

### Method of Implant

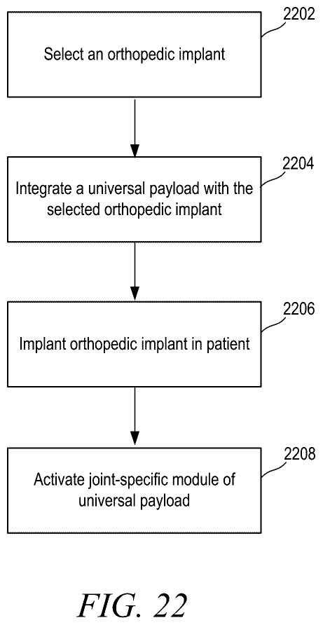

With reference to FIG. 22, the present disclosure provides a method of implanting an orthopedic implant configured to replace or functionally supplement a natural joint of a body. The orthopedic implant may be any of the orthopedic implant described herein.

At block 2202, one of a plurality of different orthopedic implants is selected for implant. Each of the different orthopedic implants has a payload receptacle configured to receive a universal payload. The payload receptacle may be any one of the payload receptacles described herein.

At block 2204, a universal payload is integrated with the selected orthopedic implant. The universal payload may be any one of the universal payloads described herein. To this end, the universal payload is inserted into the payload receptacle of the orthopedic implant and is secured to the orthopedic implant. In some embodiments the universal payload includes an electronics assembly having at least one sensor that is aligned to sense relative to an axis of a coordinate system. In this case, the universal payload is aligned with the payload receptacle of the selected orthopedic implant while the universal payload is being inserted into the payload receptacle to thereby align the axis of the sensor with a corresponding axis of the orthopedic implant.

At block 2206, the orthopedic implant is implanted in the patient. In some embodiments, the universal payload is integrated with orthopedic implant and then the orthopedic implant is implanted in the patient. In other words, the integrating of block 2204 precedes the implanting of block 2206. This order may occur, for example, when the orthopedic implant is a tibial component of a TKA system. In other embodiments, the orthopedic implant is implanted in the patient and then the universal payload is inserted in the orthopedic implant. In other words, the implanting of block 2206 precedes the integrating of block 2204. This order may occur, for example, when the orthopedic implant is a femoral component of a THA system.

In some embodiments the universal payload includes a controller having at least two joint-specific modules, each respectively configured to collect kinematic data resulting from movement of a respective specific joint sensed by a sensor of the universal payload. In this case, at block 2208, a select one of the at least two joint-specific modules is activated. For example, in some embodiments of the universal payload, there may be three joint-specific modules—one for the knee, one for the hip, one for the shoulder. Within each of these modules there is a left submodule and a right submodule. Thus, there are six options to activate—a right knee, left knee, left hip, right hip. Left shoulder, right shoulder.

### Method of Manufacture

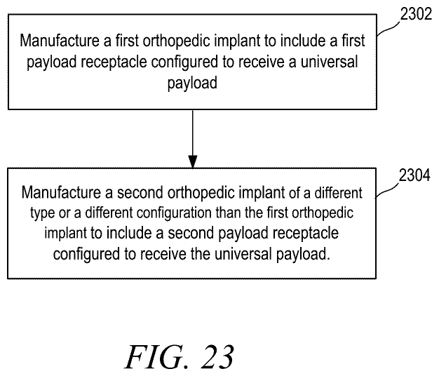

With reference to FIG. 23, the present disclosure provides a method of manufacturing a variety of different types of orthopedic implants, each configured to be integrated with a universal payload. In this way, a single universal payload can be integrated with each of the two different orthopedic implants.

At block 2302, a first orthopedic implant is manufactured to include a first payload receptacle configured to receive a universal payload. The universal payload may be any one of the universal payloads disclosed herein. The first payload receptacle may be formed into the first orthopedic implant by a molding process. Alternatively, the first payload receptacle may be drilled or machined into a solid structure, e.g., a stem of a femoral component of a hip implant system, of the first orthopedic implant. To receive a universal payload the first payload receptacle has a first receptacle form factor that is shaped and sized similar to the universal payload—but not necessarily identical to the universal payload. For example, with reference to the universal payload 100 shown in FIG. 1A, a first receptacle form factor configured to receive the universal payload would have depth as least equal to (and possibly greater than) the length L. The first receptacle form factor would also have cross-section shapes and dimensions, e.g., circular cross-sections and diameters, slightly greater than diameters D₁ and D₂ of the universal payload 100 in FIG. 1A so the payload fits within the first payload receptacle.

At block 2304, a second orthopedic implant of a different type or a different configuration than the first orthopedic implant is manufactured to include a second payload receptacle configured to receive the universal payload. To this end, in some embodiments the second payload receptacle has a second receptacle form factor that is identical to the first receptacle form factor. For example, the second receptacle form factor has the same depth and cross-section shapes and dimensions, e.g., circular cross-sections and diameters, as the first receptacle form factor. In some embodiments, the second payload receptacle has a second receptacle form factor that is similar to—but not identical to—the first receptacle form factor. For example, the second receptacle form factor may have the same cross-section shapes and dimensions, e.g., circular cross-sections and diameters, as the first receptacle form factor but a depth different than the first receptacle form factor. The second payload receptacle may be formed into the second orthopedic implant by a molding process. Alternatively, the second payload receptacle may be drilled or machined into a solid structure, e.g., a stem of a tibial component of a knee implant system, of the second orthopedic implant.

In some embodiments, the first and second orthopedic implants are configured for implantation into different joints, e.g., knee and hip, or knee and shoulder, or hip and shoulder. For example, the first orthopedic implant may be a component of a knee implant system (e.g., a tibial plate with a tibial stem, a femoral component, a tibial extension) and the second orthopedic implant may be a component of a hip implant system (e.g., a femoral component). In some embodiments, the first orthopedic implant may be a spinal implant (e.g., a rod, a pedicle screw, a spinal fusion device, a spinal cage, an artificial disc, a spinal plate).

In some embodiments, the first and second orthopedic implants are a same component of a joint implant system or a spinal implant system but are of different configurations (e.g., assorted sizes). For example, the first orthopedic implant and the second orthopedic implant may be a same component of a knee implant system (e.g., a tibial plate with a tibial stem, a femoral component, a tibial extension), or they may each be a same type of spinal implant. When the first and second orthopedic implants are a same component of a joint implant system or a same spinal implant but are of different configurations, they nevertheless are not identical to one another, but will differ in some degree. For example, they may have different sizes, however the respective payload receptacles of the first and second orthopedic implants are configured so that the same universal payload can be integrated with each of the first orthopedic implant and the second orthopedic implant.

In some embodiments, the first orthopedic implant and the second orthopedic implant are further manufactured to include the same or identical securing mechanisms or features that are compatible with the securing mechanisms or features of the universal payload. In some embodiments, the first orthopedic implant and the second orthopedic implant are further manufactured to include the same or identical alignment mechanisms or features that are compatible with the alignment mechanisms or features of the universal payload.

As mentioned previously, in some embodiments a universal payload includes a housing, at least one sensor aligned within the housing relative to an axis of a coordinate system of the universal payload to sense movement relative to the axis, and a payload alignment feature. In one embodiment, the universal payload has a payload form factor that is configured to be at least partially inserted into a payload receptacle of each of a plurality of different orthopedic implants. The payload alignment feature is configured to engage an implant alignment feature of each of the plurality of different orthopedic implants to thereby align the coordinate system of the universal payload with a corresponding coordinate system of an orthopedic implant during insertion of the universal payload into the payload receptacle.

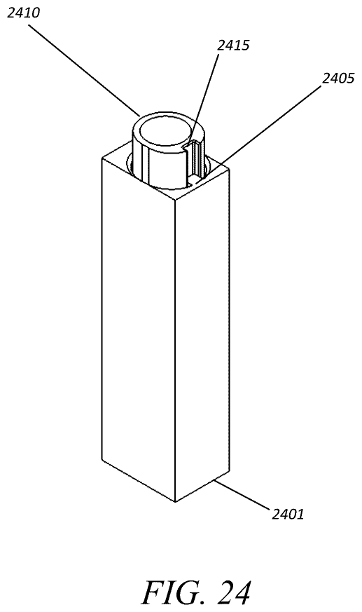

Optionally, the payload alignment feature may comprise a tab that protrudes from a surface of the universal payload, and the implant alignment feature may comprise a slot that is recessed from a surface of the implant, where upon engagement of the universal payload with the implant, the tab fits into and engages with the slot. See, e.g., FIG. 1A through FIG. 19C. Alternatively, as illustrated in FIG. 24, the universal implant 2401 (shown generically as a block) may comprise an implant alignment feature that may comprise a tab 2405 that protrudes from a surface of the receptacle of the implant, and the universal payload 2410 (shown generically as a cylinder that fits into a correspondingly shaped receptacle of the implant) may comprise a universal payload alignment feature that may comprise a slot 2415 that is recessed from a surface of the universal payload, where upon engagement of the universal payload with the implant, the tab 2405 of the implant alignment feature fits into and engages with the slot 2415 of the universal payload alignment feature. Optionally, and in any of the embodiments disclosed herein, either of the universal payload or the implant may have a tab which engages with a slot on an implant or a universal payload, respectively, to create and secure a desired alignment therebetween.

### Prosthetics

A prosthesis or prosthetic as used in the present disclosure is a component or combination of components that replaces a missing limb, e.g., hand, arm, foot, or leg, of a person or animal. Prosthetics, which may also be referred to as artificial limbs, are external devices (as opposed to implantable devices) that are attached to a limb remnant of a person. Example of such prosthetics are described in WO 2005/110293, WO 2005/117749, WO 2008/005424, WO 2009/120637, WO 2012/166853, and U.S. Pat. No. 8,632,607, which are incorporated by reference.

Like the orthopedic implants disclosed herein, it may be desirable to collect kinematic data relevant to movement of an artificial limb and by extension—the limb remnant to which the artificial limb is attached. Accordingly, the universal payload disclosed herein may also be configured to be integrated with various types, e.g., hand, arm, foot, or leg, and sizes of prosthetics that are themselves configured to receive a same universal payload.

The devices, methods, systems etc. of the present disclosure have been described broadly and generically herein. Each of the narrower species and subgeneric groupings falling within the generic disclosure also form part of the present disclosure. This includes the generic description of the devices, methods, systems etc. of the present disclosure with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the present disclosure are described in terms of Markush groups, it is intended, and those skilled in the art will recognize, that the present disclosure embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. For example, the term “a sensor” refers to one or more sensors, and the term “a medical device comprising a sensor” is a reference to a medical device that includes at least one sensor. A plurality of sensors refers to more than one sensor. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include,” as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the disclosure, invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the disclosure, invention, or claims. The upper and lower limits of these smaller ranges may independently be included in the smaller ranges, subject to any specifically excluded limit in the stated range. Where the stated range includes one or both of the limits, ranges excluding either or both of those included limits are also included in the disclosure.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

As mentioned above, in the following claims, the terms used should not be construed to limit the claims to the specific embodiments disclosed in the specification and the claims, but should be construed to include all possible embodiments along with the full scope of equivalents to which such claims are entitled. For example, described embodiments with one or more omitted components or steps can be additional embodiments contemplated and covered by this application.

1. A universal payload comprising:
an electronics assembly comprising at least one sensor, and a controller having at least two joint-specific modules, each joint-specific module respectively configured to collect kinematic data resulting from movement of a respective specific joint sensed by the at least one sensor; and
a housing enclosing the electronics assembly; and
a payload coupling feature configured to couple to each of a plurality of different orthopedic implants configured to replace or functionally supplement a natural joint of a body.

2. The universal payload of claim 1, wherein the at least two joint specific modules comprise two or more of:
a knee module configured to collect kinematic data resulting from movement of a knee sensed by the at least one sensor,
a hip module configured to collect kinematic data resulting from movement of a hip sensed by the at least one sensor, and
a shoulder module configured to collect kinematic data resulting from movement of a shoulder sensed by the at least one sensor.

3. The universal payload of claim 1, wherein each of the at least two joint-specific modules is configured to be placed in one of an active state and an inactive state.

4. The universal payload of claim 1, wherein the at least one sensor comprises one or more accelerometers, one or more gyroscopes, or a combination of one or more accelerometers and one or more gyroscopes.

5. The universal payload of claim 1, further comprising an antenna assembly coupled to the electronics assembly and extending from an end of the housing.

6. The universal payload of claim 5, wherein:
the antenna assembly comprises an antenna that couples to the electronics assembly, and a cap that covers the antenna; and
the universal payload further comprises an external sensor associated with the cap and that couples to the electronics assembly.

7. An orthopedic implant comprising:
an implant component configured to extend at least partially into a bone of a body; and
a universal coupling feature associated with the implant component, the universal coupling feature being identical to a coupling feature in each of a plurality of different implant components, and,
wherein the universal coupling feature is configured to couple to a payload coupling feature of a universal payload having an electronics assembly comprising at least one sensor, and a controller having at least two joint-specific modules, each joint-specific module respectively configured to collect kinematic data resulting from movement of a respective specific joint sensed by the at least one sensor.

8. The orthopedic implant of claim 7, wherein the implant component comprises one of a tibial stem, a tibial stem extension, a humeral stem, and a femoral stem.

9. An intelligent implant comprising:
an orthopedic implant having a universal coupling feature identical to a coupling feature of each of a plurality of different orthopedic implants; and
a universal payload comprising:
an electronics assembly comprising at least one sensor, and a controller having at least two joint-specific modules, each joint-specific module respectively configured to collect kinematic data resulting from movement of a respective specific joint sensed by the at least one sensor,
a housing enclosing the electronics assembly, and
a payload coupling feature configured to couple to the universal coupling feature.

10. The intelligent implant of claim 9, wherein the orthopedic implant is a component of one of a knee implant, a hip implant, and a shoulder implant.

11. The intelligent implant of claim 9, wherein:
the at least one sensor is aligned within the universal payload relative to an axis of a coordinate system of the universal payload to thereby sense movement relative to the axis; and
the universal payload and the universal coupling feature have complimentary alignment features that engage to align the coordinate system of the universal payload with a corresponding coordinate system of the orthopedic implant during coupling of the universal payload with the universal coupling feature.

12. The intelligent implant of claim 9, wherein the universal payload and the universal coupling feature have corresponding securing features that function to secure the universal payload relative to the orthopedic implant during coupling of the universal payload with the universal coupling feature.

13.-88. (canceled)
